(19) United States
(12) Patent Application Publication
(10) Pub. No.: US 2022/0008250 A1
(43) Pub. Date: Jan. 13, 2022

(54) DEVICES AND METHODS FOR TARGETED DELIVERY OF MATERIAL

(71) Applicant: Sanulus Medical, LLC, Chicago, IL (US)

(72) Inventors: John S. Pollack, Naperville, IL (US); William R. Voss, Hinsdale, IL (US)

(21) Appl. No.: 17/368,551

(22) Filed: Jul. 6, 2021

Related U.S. Application Data
(60) Provisional application No. 63/050,695, filed on Jul. 10, 2020.
(60) Provisional application No. 63/049,044, filed on Jul. 7, 2020.

(51) Int. Cl. A61F 9/007

(52) U.S. Cl. CPC A61F 9/00727; A61B 17/00491

(57) ABSTRACT

Disclosed herein are embodiments of devices and systems for applying a bioadhesive substance to a retina that can include an outer sleeve having a proximal end, a distal end, a passageway extending along a length of the elongate body and an opening at the distal end of the elongate body, an applicator tip including an elongate body having a proximal end and a distal end, an applicator portion coupled with the distal end of the elongate body, and a substance supply lumen extending along a length of the device through which a bioadhesive substance can pass so that the bioadhesive substance can be applied to at least the applicator portion of the applicator tip. Some embodiments of the device can also have a source of the bioadhesive substance in fluid communication with at least the substance supply lumen.

### PRIORITY CLAIM AND INCORPORATION BY REFERENCE

The present application claims the benefit under 35 U.S.C. § 119(e) to U.S. Patent Application No. 63/049,044, filed on Jul. 7, 2020 and U.S. Patent Application No. 63/050,695 filed on Jul. 10, 2020. The contents of each of these priority applications are hereby incorporated by reference herein in their entirety as if fully set forth herein for all purposes. Any and all applications for which a foreign or domestic priority claim is identified in the Application Data Sheet as filed with the present application are hereby incorporated by reference herein in their entirety and made a part of this specification.

### FIELD

Devices and methods disclosed herein relate to targeted delivery of a substance to a tissue surface, such as the delivery and application of a bioadhesive to a retinal tissue.

### BACKGROUND

Existing methods for fixing macular holes and retinal detachments, associated with retinal discontinuities such as tears and holes, include perfluorocarbon, laser, cryotherapy, gas bubble injection, and the use of silicone oil. The use of perfluorocarbon for displacement of fluid out of the subretinal space may result in inadvertent migration of perfluorocarbon liquid into the subretinal space which can result in vision loss if it is located in the central macula. Incomplete removal of perfluorocarbon liquid from the vitreous cavity may result in associated visual phenomena that patients may find annoying. Use of laser and cryotherapy results in permanent retinal scar tissue formation and may contribute to epiretinal membrane and epiretinal scar tissue formation. Use of gas bubble injection results in limitations on activities of the patient and can cause complications such as elevated intraocular pressure and cataracts. Likewise, silicone oil can be problematic because it sometimes causes complications such as elevation of intraocular pressure, requires a second surgery for removal, and frequently leaves behind residual oil bubbles after attempted removal that patients may find visually distracting.

Existing methods for fixing leaking eye wall discontinuities such as incisions, sclerotomies, and lacerations include sutures and commercially available bioadhesives. Sometimes sutured discontinuities continue to leak. Sutures cause ocular irritation, pain, tearing, and sometimes local tissue reactions that included edema and inflammation of the episclera and conjunctiva.

One difficulty with some bioadhesives is that they may induce toxicity. Another difficulty is that bioadhesive can be difficult to apply to non-dependent ocular surfaces, both inside and outside the eye, due to the effect of gravity on the bioadhesive as it leaves the tip of the bioadhesive delivery device. For example, in an eye with a retinal tear that is located on the lateral or superior aspect of the eye, gravity may make it difficult to apply the bioadhesive to the retinal tear since gravity may cause dripping or drooping of the bioadhesive as soon as it is expelled from the applicator device, preventing its accurate application over the retinal discontinuity. The same problem occurs when attempting to apply liquid or viscous materials to lateral and inferior outer eye surfaces. Another difficulty with bioadhesives is that they can be difficult to apply to large discontinuities of retina and other ocular surfaces, as well as tissue discontinuities that have an irregularly shaped margin or margins that are at different elevations from one another. Difficulties applying bioadhesives to retinal discontinuities is even more difficult if attempted using a transcleral, subretinal approach.

### SUMMARY OF SOME EXEMPLIFYING EMBODIMENTS

The systems, methods and devices of this disclosure each have several innovative aspects, implementations, or aspects, no single one of which is solely responsible for the desirable attributes disclosed herein.

Disclosed herein are embodiments of devices and systems for applying a bioadhesive substance to a retina including an outer sleeve having a proximal end, a distal end, a passageway extending along a length of the elongate body, and an opening at the distal end of the elongate body, an applicator tip including an elongate body having a proximal end and a distal end, an applicator portion coupled with the distal end of the elongate body, and a substance supply lumen extending along a length of the device through which a bioadhesive substance can pass so that the bioadhesive substance can be applied to at least the applicator portion of the applicator tip. Some embodiments of the device can also have a source of the bioadhesive substance in fluid communication with at least the substance supply lumen.

Any embodiments of the devices, systems, and methods disclosed herein can include, in additional embodiments, one or more of the following features, components, and/or details, in any combination with any of the other features, components, and/or details of any other embodiments disclosed herein: wherein the applicator tip is configured to move between a first position wherein the applicator portion is contained within the outer sleeve and a second position wherein the applicator tip is positioned past the opening at the distal end of the outer sleeve; wherein the applicator portion can have a first width when the applicator portion is in the first position and a second width when the applicator portion is the second position, the second width being substantially greater than the first width; wherein the substance supply lumen is a passageway extending through at least the elongate body of the applicator tip; wherein the substance supply lumen can include a separate cannula positioned within the outer sleeve and extendable and retractable relative to the outer sleeve; wherein the substance supply lumen can be axially fixed relative to the outer sleeve and can have an opening that is adjacent to an end portion of the outer sleeve such that the bioadhesive substance can be exuded onto the applicator portion along at least a portion of a length of the applicator portion as the applicator portion is advanced distally; wherein the device includes a handle coupled with a proximal end of the outer sleeve; wherein the applicator portion can be biased such that a first side portion rolls toward a second side portion of the applicator portion when the applicator tip is withdrawn in the proximal direction into the outer sleeve; wherein the elongate body of the applicator tip extends through the outer sleeve to at least a proximal end of the device; wherein the elongate body of the applicator tip can be integrally formed with the applicator portion; wherein the applicator portion can be nonremovably coupled with the elongate body of the applicator tip; wherein the applicator portion can be configured to be removed from the patient's body with the device; wherein the applicator portion can be configured to be moved to the first position before the device is removed from the patient's body; wherein the applicator portion can be biased to collapse within the passageway of the outer sleeve when the applicator portion is withdrawn in the proximal direction into the outer sleeve; wherein the applicator portion can be biased such that a first side portion folds toward a second side portion of the applicator portion when the applicator tip is withdrawn in the proximal direction into the outer sleeve; wherein the applicator portion can be biased such that a first side portion collapses toward a second side portion of the applicator portion when the applicator tip is withdrawn in the proximal direction into the outer sleeve; and/or wherein the passageway through at least the elongate body of the applicator tip is in communication with an opening at a proximal end of the applicator portion.

Any embodiments of the devices, systems, and methods disclosed herein can include, in additional embodiments, one or more of the following features, components, and/or details, in any combination with any of the other features, components, and/or details of any other embodiments disclosed herein: wherein the passageway through at least the elongate body of the applicator tip is in communication with a plurality of openings passing through a first surface of the applicator portion; wherein the applicator portion can have an asymmetric shape such that a first side portion on one side of an axial centerline of the applicator portion can have a different shape than a second side portion on an opposite side portion of the applicator portion, the first and second sides being divided an axial centerline of the applicator portion; wherein the applicator portion can have an asymmetric shape having a first side portion on one side of an axial centerline of the applicator portion that can have a first length that can be longer than a second length of a second side portion on a second side of the axial centerline of the applicator portion; wherein the first side portion can have a first transition portion that has a first length in the axial direction and the second side portion has a second transition portion that has a second length in the axial direction, and wherein the first length of the first transition portion can be less than the second length of the second transition portion; wherein the applicator portion can have an asymmetric width; wherein the applicator portion can have an asymmetric thickness in a widthwise direction, such that a thickness profile of the applicator portion on a first side of the applicator portion can be different than a thickness profile of the application portion on a second side of the applicator portion; wherein the first side portion can be more flexible than the second side portion of the applicator portion; and/or wherein the applicator portion can be configured such that a first side portion contracts before a second side portion contracts when the applicator portion is withdrawn into the outer sleeve; wherein the applicator portion can have an asymmetric width and an asymmetric thickness.

Any embodiments of the devices, systems, and methods disclosed herein can include, in additional embodiments, one or more of the following features, components, and/or details, in any combination with any of the other features, components, and/or details of any other embodiments disclosed herein: wherein the outer sleeve can have a notch in the distal end thereof; wherein the outer sleeve can have a notch in the distal end thereof configured to facilitate contraction or reduction in size of the applicator portion as the applicator portion is being retracted into the distal end of the outer sleeve; wherein the notch can be generally in the shape of a “v” or can have a tapered shape that narrows in the proximal direction; and/or wherein the notch can be generally in the shape of a “u” or can have a uniform width and a rounded end portion at a proximal end of the notch.

Disclosed herein are embodiments of devices and systems for applying a substance to a tissue surface including a cannula comprising a proximal end, a distal end, and a passageway extending along a length of the cannula and an applicator tip. In some embodiments, the applicator tip can include an elongate body having a proximal end and a distal end, an applicator portion coupled with the distal end of the elongate body, a passageway through at least the elongate body of the applicator tip, a handle coupled with a proximal end of the cannula, and a source of the substance in fluid communication with at least the passageway extending along a length of the elongate body of the applicator tip.

Any embodiments of the devices, systems, and methods disclosed herein can include, in additional embodiments, one or more of the following features, components, and/or details, in any combination with any of the other features, components, and/or details of any other embodiments disclosed herein: wherein the applicator tip can be configured to move along a length of the passageway of the cannula between a first position wherein the applicator portion is contained within the cannula and a second position wherein the applicator tip extends past the distal end of the cannula; wherein the applicator portion can have a first width when the applicator portion is in the first position and a second width when the applicator portion is the second position, the second width being substantially greater than the first width; wherein the applicator portion can be biased to collapse within the passageway of the cannula when the applicator tip is withdrawn in the proximal direction into the cannula; wherein the substance can be a bioadhesive; wherein the tissue can include an eye tissue; wherein the applicator portion can be biased such that a first side portion rolls toward a second side portion of the applicator portion when the applicator tip is withdrawn in the proximal direction into the cannula; wherein the applicator portion can be biased such that a first side portion folds toward a second side portion of the applicator portion when the applicator tip is withdrawn in the proximal direction into the cannula; wherein the applicator portion can be biased such that a first side portion collapses toward a second side portion of the applicator portion when the applicator tip is withdrawn in the proximal direction into the cannula; wherein the passageway through at least the elongate body of the applicator tip is in communication with an opening at a proximal end of the applicator portion; and/or wherein the passageway through at least the elongate body of the applicator tip can be in communication with a plurality of openings passing through a first surface of the applicator portion.

Disclosed herein are embodiments of methods of repairing a defect in a retinal tissue, that can include advancing a device for applying a bioadhesive substance toward the defect, covering at least a portion of an applicator tip of the device with the bioadhesive substance, advancing the applicator tip past a distal end of an outer sleeve of the device, and transferring the bioadhesive substance from the applicator tip of the device to the defect. In some embodiments, the method can also include rolling or collapsing the applicator tip to a smaller size by withdrawing the applicator tip into the outer sleeve.

### BRIEF DESCRIPTION OF THE DRAWINGS

FIG. 1 depicts a top view of an embodiment of a device for application of a substance to a surface of an object, showing the device in a second or extended state.

FIG. 2 depicts a side view of the embodiment of the device shown in FIG. 1, showing the device in the second or deployed state.

FIG. 3 depicts a side view of the embodiment of the device shown in FIG. 1, showing the device in a first or withdrawn state.

FIG. 4 depicts an end view of the embodiment of the device shown in FIG. 1, showing the device in the first or withdrawn state.

FIG. 5 depicts an end view of another embodiment of the device, showing the device in the first or withdrawn state.

FIG. 6 depicts a top view of another embodiment of an applicator.

FIG. 7A depicts a section view of another embodiment of a device for application of a substance to a surface of an object, showing the device in the second or deployed state.

FIG. 7B depicts a section view of another embodiment of a device for application of a substance to a surface of an object, showing the device in the second or deployed state.

FIG. 8A depicts a non-limiting example of a system or device described herein.

FIG. 8B depicts a non-limiting example of a system or device described herein.

FIG. 8C depicts a non-limiting example of a system or device described herein.

FIG. 9 depicts an example of a cannula with a fiber or wire for light or cautery.

FIG. 10 depicts a side view of another embodiment of a device for application of a substance to a surface of an object, showing the device in a second or extended state.

FIG. 11 depicts a side view of the embodiment of a device shown in FIG. 10, also showing the device in a second or extended state.

FIG. 12 is a side view of an embodiment of a device for application of a substance to a surface that has been surgically advanced into an eye, showing the applicator portion in a retracted state.

FIG. 13 is a side view of the device show in FIG. 12 that has been surgically advanced into an eye, showing the applicator portion in an extended, expanded state.

FIG. 14 is a side view of another embodiment of a device for application of a substance to a surface that has been surgically advanced into an eye, showing a supply cannula in a retracted state within the outer cannula.

FIG. 15 is a side view of the embodiment of a device show in FIG. 14, showing the supply cannula in an extended state so as to overlap a portion of the applicator portion.

FIG. 16 is a side view of the embodiment of a device show in FIG. 14, showing the applicator portion at least partially covered with a substance.

FIG. 17 is a side view of the embodiment of a device show in FIG. 14, showing the applicator portion transferring the substance to a tissue defect in the eye.

FIG. 18A is a perspective view of an end portion of an embodiment of a cannula that can be used with any embodiments of the devices disclosed herein.

FIG. 18B is a section view of the embodiment of the cannula shown in FIG. 18A, taken through line 18B-18B of FIG. 18A.

FIG. 18C is a top view of the embodiment of the cannula shown in FIG. 18A.

FIG. 19A is a perspective view of an end portion of another embodiment of a cannula that can be used with any embodiments of the devices disclosed herein.

FIG. 19B is a section view of the embodiment of the cannula shown in FIG. 19A, taken through line 19B-19B of FIG. 19A.

FIG. 19C is a top view of the embodiment of the cannula shown in FIG. 19A.

FIG. 20A is a top view of an end portion of another embodiment of a cannula that can be used with any embodiments of the devices disclosed herein.

FIG. 20B is a top view of an end portion of another embodiment of a cannula that can be used with any embodiments of the devices disclosed herein.

FIG. 20C is a top view of an end portion of another embodiment of a cannula that can be used with any embodiments of the devices disclosed herein.

FIG. 21 is a top view of an embodiment of an applicator that can be used with any device embodiments disclosed herein.

FIG. 22 is a top view of another embodiment of an applicator that can be used with any device embodiments disclosed herein.

FIG. 23 is a top view of another embodiment of an applicator that can be used with any device embodiments disclosed herein.

FIG. 24 is a top view of another embodiment of an applicator that can be used with any device embodiments disclosed herein.

FIG. 25 is a section view of an embodiment of an applicator portion that can be used with any device embodiments disclosed herein, taken through line 25-25 of FIG. 21 so that the section view is perpendicular to the axial centerline of the applicator.

FIG. 26 is a section view of another embodiment of an applicator portion that can be used with any device embodiments disclosed herein, wherein the section view is perpendicular to the axial centerline of applicator.

FIG. 27 is a section view of another embodiment of an applicator portion that can be used with any device embodiments disclosed herein, wherein the section view is perpendicular to the axial centerline of applicator.

FIG. 28 is a section view of another embodiment of an applicator portion that can be used with any device embodiments disclosed herein, wherein the section view is perpendicular to the axial centerline of applicator.

FIG. 29 is a section view of another embodiment of an applicator portion that can be used with any device embodiments disclosed herein, wherein the section view is perpendicular to the axial centerline of applicator.

FIG. 30 is a perspective view of an end portion of another embodiment of a delivery device, showing an applicator in a retracted and collapsed state and a substance delivery cannula in a retracted state.

FIG. 31 is a perspective view of the end portion of the embodiment of a delivery device shown in FIG. 30, showing the applicator portion in a partially advanced position and expanded state.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

There is a need in the art for non-problematic, easy-to-use devices and methods for applying therapeutic substances to ocular or other biological tissue. Disclosed herein are systems, devices, and methods for applying a substance to a surface, such as for example and without limitation, for applying a bio substance to a tissue surface. Some Nonlimiting examples of applications of any of the devices and methods disclosed herein include applying a bio-substance such as a bioadhesive to an ocular tissue such as for a retinal tear, hole, or detachment, and applying bioadhesive to the sclera, conjunctiva, and cornea, applying a bio-substance such as a bioadhesive to gastrointestinal tissue, colon tissue, etc. for sealing perforations or tears therein. However, the embodiments of the devices and methods disclosed herein are not limited to the delivery of therapeutic substances or ocular, medical, or biological applications. The devices and methods disclosed herein can be used for the delivery of any desired or suitable material in any desired application—biological, non-biological, mechanical, or otherwise.

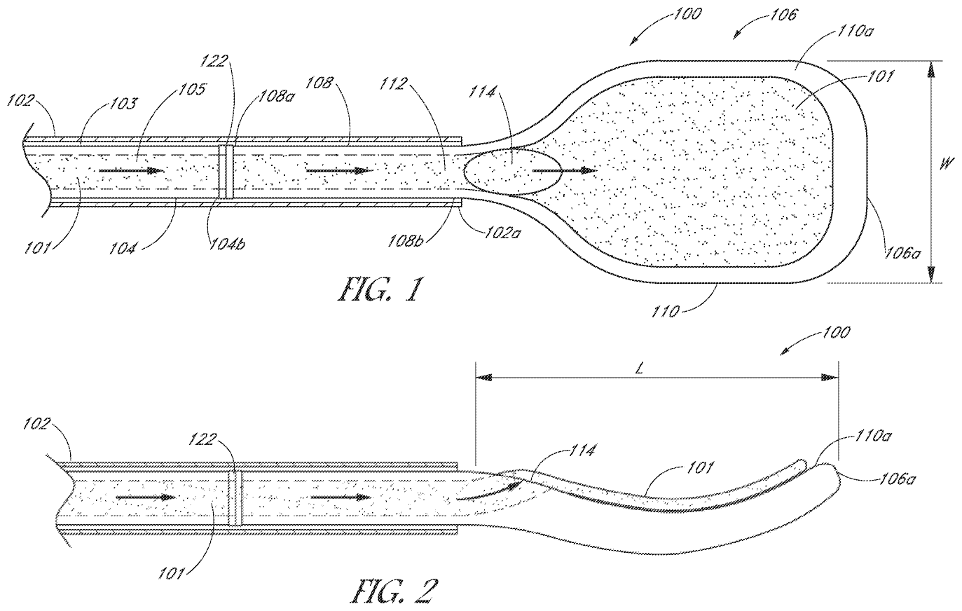

FIGS. 1 and 2 depict a top view and a side view, respectively, of an embodiment of a device 100 (also referred to herein as a delivery device) for application of a substance 101 to a surface of an object. In some embodiments, the device 100 can include a cannula or outer sleeve 102 (also referred to herein as an elongate body or outer sheath), an inner sleeve 104 (also referred to herein as an inner cannula) having a lumen or passageway 105 therein through which the substance 101 can be advanced, and an applicator 106 (also referred to herein as an applicator tip) coupled with the inner sleeve 104. The outer sleeve 102 can be configured to provide additional rigidity and manipulability to the inner sleeve 104 and at least a portion of the applicator 106 to facilitate or improve the user's control of the device 100 during application of the substance 101.

The inner sleeve 104 can have a proximal end 104a (not shown) and a distal end 104b. The passageway 105 can extend from the proximal end 104a to the distal end 104b of the inner sleeve 104. As shown, the applicator 106 can be coupled with the distal end 104b of the inner sleeve 104.

The cannula can optionally be flexible, semi-rigid, or rigid and can be formed from any suitable materials, including metal alloys, polymer materials, or otherwise. The system can optionally be configured to not have an outer sleeve 102, wherein the inner sleeve 104 and the applicator 106 are sufficiently rigid and manipulable to not require the outer sleeve 102.

In any embodiments disclosed herein, the applicator 106 and/or other components may be pre-loaded in the cannula or outer sleeve 102 such that the applicator 106 need not be advanced into a proximal end of the cannula 102 after the cannula 102 has been advanced to the target location. Optionally, the device 100 can be configured such that the applicator 106 is advanced into the proximal end of the cannula 102 after the cannula 102 has been advanced into the target location. A proximal end of the cannula 102 can be tapered to facilitate the insertion of the applicator portion 110 into the cannula 102 and/or facilitate the collapse and/or rolling of the applicator portion 110 for insertion into the cannula 102.

In any embodiments of the device 100, the handle, cannula or outer sheath 102, the inner sheath 104, or other rigid or semi-rigid components can be made from or comprise a any suitable plastic material, metal, polyvinyl chloride, glass, acrylic, carbon fibers and/or any combination of the foregoing. In some embodiments, the cannula tip plug or protective tip cover can comprise plastic, metal, polyvinyl chloride, glass, acrylic, carbon fibers, rubber (such as, without limitation, silicon), and/or any combination of the foregoing.

A distal end portion 106a of the applicator 106 can extend out of a distal end 102a of the cannula 102. In any embodiments, the applicator 106 can have an elongate portion 108 and an applicator portion 110. The elongate portion 108 of the applicator 106 can optionally have a passageway 112 extending therethrough from a proximal end 108a to a distal end 108b of the elongate portion 108. The passageway 112 can, in an operable state, be in fluid communication at the proximal end 108a of the passageway 108 with the passageway 105 of the inner sleeve 104. An opening or orifice 114 can be positioned at a distal end 108b of the body 108 and can be in fluid communication with the passageway 112. Some embodiments can have a connector 122 that couples the proximal end 108a of the body 108 with the distal end 104b of the inner sleeve 104. In this embodiment, the substance 101 can be advanced through the passageway 105 of the inner sleeve 104, through the connector 122, through the passageway 112 extending through the elongate portion 108 of the applicator 106, and be expressed out through the opening 114 onto a first surface 110a of the applicator portion 110 of the applicator 106.

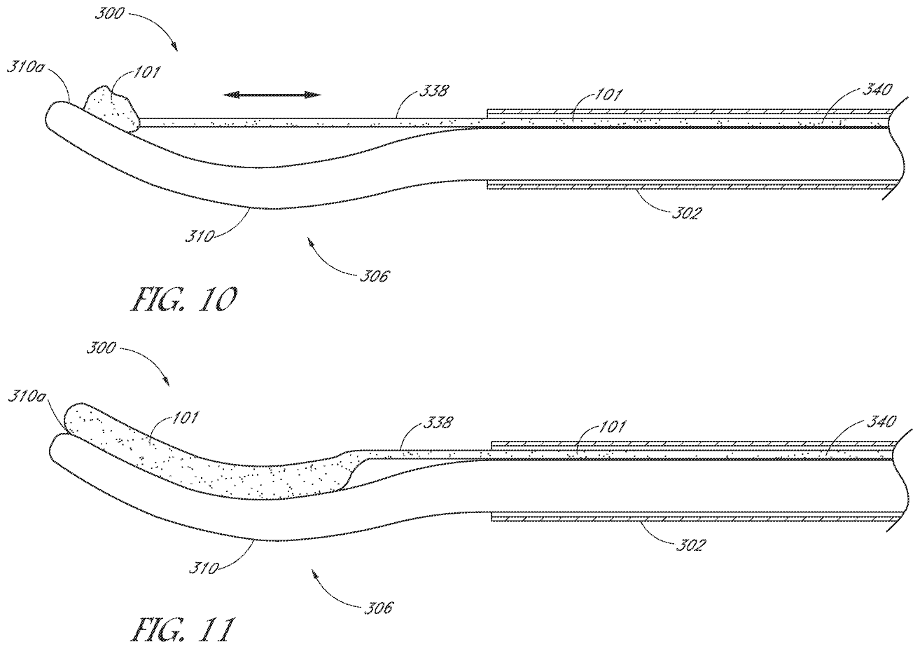

In another embodiment, as shown in FIGS. 10 and 11, the device 300 can have an outer sleeve 302 and an applicator portion 310, and an additional movable cannula 338 having a passageway 340 positioned within the outer sleeve or cannula 302. The passageway 340 can optionally provide a passageway for the transmission or provision of substance 101 (which can, optionally, be the substance described above or in any other embodiments disclosed herein or any other desired material) onto various parts of applicator portion 310. As will be described, the movable cannula 338 (also referred to herein as a fill or refill cannula) can be configured to be moveable relative to the outer sleeve 302, and/or at least a portion of the applicator 306 from at least a first position, as illustrated in FIG. 10, to a second position, as illustrated in FIG. 11. The applicator 306 and applicator portion 310 can have any of the features, materials, or other details of any of the other applicators and applicator portions disclosed herein, including without limitation applicator 106 and applicator portion 110.

In any embodiments disclosed herein, the device can have an outer sleeve and an applicator portion, and an additional fixed lumen or cannula (such as, for example, a fixed version of cannula 338) having a passageway therein within the outer sleeve or cannula. The lumen or cannula can provide a passageway for the transmission or provision of substance 101 (which can, optionally, be the substance described above or in any other embodiments disclosed herein, or any other desired material) onto the applicator portion 310. For example and without limitation, the substance can be exuded through an opening at a distal end of the lumen or cannula and onto the applicator portion. Further, some embodiments of the device can be configured such that the substance can be first exuded on a distal or leading edge of the applicator portion, and then across a length of the applicator portion as the applicator portion is extended in the distal direction, as substance is continuously injected and exuded through the opening at the distal end of the substance supply lumen or passageway. In some embodiments, the distal end of the substance supply lumen or cannula can be configured to exude the substance in a wide band on the applicator portion, such as through multiple openings, a manifold, or a wide slot opening. In some embodiments, the only movable or extendable component of the device or a distal end portion of the device can be the applicator and applicator portion (which can be integrally connected).

In practice, after the applicator 306 is advanced to its fully extended position, the cannula 338 can be advanced to, for example and without limitation, the distal portion of the applicator portion 310, i.e., the first position shown in FIG. 10. The substance 101 can be advanced through the passageway 340 and onto the distal portion 310a of the applicator portion 310 first. Then, as the cannula 338 is retracted (which can, optionally, be done slowly for better control and coverage of the applicator portion 310) toward the second position (as shown in FIG. 11), the user can continue to eject substance on middle portions and/or the proximal portion of the applicator portion 310. In this configuration, the user (who can optionally be a surgeon or other medical practitioner) can achieve a more even consistency and distribution of the substance over some or all portions (distal, medial, and proximal portions) of the applicator portion 310 using the moveable cannula. In some embodiments, the movable cannula can enable a more even consistency and distribution of the substance over some or all portions (distal, medial, and proximal portions) of the applicator portion 310 as compared to a fixed passageway or cannula.

Additionally, in any embodiments disclosed herein, the device can have a first or fixed inner cannula (such as cannula 104) and a moveable cannula surrounding an outside surface of the inner cannula 104, which is advanceable as described above through the passageway through the distal tip. In these embodiments, two passageways can be used to provide a substance to the applicator portion. The moveable passageway, which can optionally be an additional cannula or sleeve, can be configured to be advanceable and retractable relative to the inner cannula 104, passageway through the applicator, and/or the applicator portion.

Thereafter, once the substance 101 has been advanced onto the first surface of the applicator portion 110 (or 310) of the applicator 106, the user can apply the substance 101 to the desired surface. For example, in applications where the device 100 is used for application of a therapeutic substance to an ocular surface, the surgeon or medical practitioner can advance the substance 101 onto the first surface 110a of the applicator portion 110 of the applicator 106 in there after use the applicator portion 110 to apply the substance 101 to the target portion of the ocular surface. In any embodiments, the applicator 106 can be made from or otherwise configured to be flexible and conformable to reduce the risk of any puncture or other damage to the tissue to which the substance 101 is applied. In some embodiments, the applicator 106 can be made from a medical grade silicone material, one or more biodegradable materials, one or more different types of alloys, including stainless steel and Nitinol, or any other suitable rubber or other flexible polymeric materials. In any embodiments, the applicator portion 110 can be made from a material designed to enhance the thermal conductivity of the applicator portion 110, i.e., from a material that has enhanced thermal conductivity properties to better enable the applicator portion 110 to transfer heat or cooling.

Any embodiments of the device 100 can optionally have a distal tip (not shown) coupled with a distal end 102a of the outer sheath or cannula 102. The distal tip can optionally have a conical or tapered profile, and can be made from a flexible material such as silicone or other suitable polymers, alloys such as stainless steel or Nitinol, etc. Alternatively, in other embodiments, the distal end 102a of the cannula 102 can have an angled or beveled end surface configured to facilitate the advancement of the distal end 102a through an opening in a tissue surface or other opening.

In other embodiments the distal end 102a may be pointed and sharp, like a hollow needle tip, to allow insertion through tissue, such as through the sclera to access the subretinal space. Optionally, the device of any embodiments disclosed herein can have a distal tip portion distal to the cannula 102 comprising a softer material, such as a flexible silicone. The soft tip portion can be used in some applications to reduce the risk of trauma to the tissue surface. Additionally, in any embodiments, the soft tip portion can also be used to brush the surface of the tissue to gently spread or move the substance, remove substance from the tissue, or otherwise.

The device 100 can also optionally have a removable distal plug or tip cover that can be removably coupled with a distal end 102b of the cannula 102 and/or a distal tip of the device 100. The distal plug or tip cover can provide a seal with the passageway through the cannula 102, and can be removed prior to advancement of the device into the target tissue area to provide a seal for the device to maintain sterility within the device and also for sealing off the passageway through the cannula in devices that are pre-filled with sub stance.

Additionally, as most clearly shown in FIG. 2, some embodiments of the applicator 106 can have a curved profile in at least one of a length direction L (identified in FIG. 2) and a width direction W (identified in FIG. 1). For example and without limitation, some embodiments of the applicator portion 110 can have a curved profile or slightly curved profile in both the lengthwise direction and the widthwise direction, wherein the curved profile is sized and configured to be similar to a curvature of the target tissue surface (which can, optionally, be a retinal surface or other ocular surface of a human eye, an eye of an animal or living being, or otherwise) and/or to better hold the desired substance on the applicator portion. In other embodiments, the applicator portion 110 can be configured to improve the ability of the applicator portion 110 to hold the substance 101 on the applicator portion 110, particularly when the applicator portion 110 is rotated, tilted, and/or turned upside down. In other embodiments, the applicator portion 110 can have a generally flat profile or shape, or any other desired profile or shape.

The cannula 102 and/or cannula (or inner sleeve) 104 of any embodiments disclosed herein can optionally have a tubular shape and a size that is approximately 25 gauge (0.455 mm size), or 27 gauge (0.361 mm size), or that is from or from approximately 28 gauge (0.33 mm size) or smaller to or to approximately 20 gauge (0.813 mm) or larger, or from 27 gauge (0.361 mm size) to or to approximately 24 gauge (0.511 mm), or of any values or from and to any values within these ranges. Optionally, in other embodiments, larger cannula or smaller cannula can be used for the inner and/or outer cannula or sleeves.

In any embodiments, the cannula, cannula base, cannula body, and/or cannula tip can have a 0.1-0.5 mm, 0.5-1 mm, 1-2 mm, 2-3 mm, 3-4 mm, 4-5 mm, 5-6 mm, 6-7 mm, 7-8 mm, 8-9 mm or 9-10 mm cross-sectional diameter. Optionally, the cannula, cannula base, cannula body, cannula tip and/or cannula exit port can have a length of 0.1-0.5 mm, 0.5-1 mm, 1-2 mm, 2-3 mm, 3-4 mm, 4-5 mm, 5-6 mm, 6-7 mm, 7-8 mm, 8-9 mm, 9-10 mm, 10-20 mm, 20-30 mm, 30-40 mm or 40-50 mm. Additionally, the cannula, cannula base, cannula body, cannula tip and/or cannula exit port can have an external diameter, an internal diameter and a width between the internal diameter and the external diameter of 0.01-0.05 mm, 0.05-0.1 mm, 0.1-0.5 mm, 0.5-1 mm, 1-2 mm, 2-3 mm, 3-4 mm, 4-5 mm, 5-6 mm, 6-7 mm, 7-8 mm, 8-9 mm, 9-10 mm, 10-20 mm, 20-30 mm, 30-40 mm or 40-50 mm.

For other applications outside of ocular applications, the cannula 102 can optionally have a tubular shape and a cross-sectional size or inner diameter that is from or from approximately 10 mm to or to approximately 200 mm or more, or from or from approximately 40 mm to or to approximately 80 mm.

Some embodiments of the devices and methods disclosed herein can be configured to deliver and apply a variety of materials or substances of varying viscosities to the target tissue or surface. Examples of substances that can be used with any of the devices, systems, and methods disclosed herein include, without limitation, adhesives, bioadhesives, gels, hydrogels, thick liquids or semi-liquid treatment substances, double layer hydrogels, nonsolids, a polyethylene glycol solutions, a trilysine amine solutions, polymeric hydrogels, thermoresponsive gels, and any combination of the foregoing. Examples of bioadhesive substances that can be used in any embodiments disclosed herein includes, without limitation, ReSure™ sealant, a polyethylene glycol hydrogel and other hydrogels, cyanoacrylate, fibrin glue, a polyethylene glycol solution, trilysine amine solution, glycoproteins, elastic proteins, carbohydrates, mucopolysaccharides, etc. In some embodiments of the system or device, the bioadhesive material comprises a resin. UV or light activated bioadhesives can also optionally be used with any device embodiments disclosed herein. In some such embodiments, the system, kit or device can incorporate a light or light fiber for activating the bioadhesive material.

Temperature activated bioadhesives can also optionally be used with any device embodiments disclosed herein. In some such embodiments, the system, kit or device can incorporate a material that conducts heat for activating the bioadhesive material.

In some embodiments, the substance can include an adhesive or bioadhesive. The term, “adhesive,” as used herein, shall be given its ordinary meaning and also is intended to encompass a bioadhesive. Examples of adhesives that can be used with any embodiments disclosed herein include but are not limited to polyvinyl acetate, glue, aliphatic, cyanoacrylate, epoxy, polyurethane glue, and contact cement. Examples of bioadhesives include but are not limited to. In some embodiments the adhesive is a biomimetic material. In some embodiments, the adhesive is not a bioadhesive.

Any embodiments disclosed herein can also be configured to use one or more substances that polymerize as the temperature of the substance reaches a threshold value or range of values, such as for example and without limitation, normal body temperature, or to any values from 80° F. or from approximately 80° F. to or to approximately 100° F. or greater, or from or from approximately 90° F. to 100° F. or to approximately 100° F. or greater, or from 95° F. or from approximately 95° F. to 100° F. or to approximately 100° F. or greater, or to and from any values within these ranges. Additionally, any embodiments can be used to apply a cold cured substance to a tissue surface—i.e., substances that polymerize as the temperature of the substance drops to a threshold value or range of values, such as for example and without limitation, 10° F. below body temperature, or to any values from 70° F. or from approximately 70° F. or less to 90° F. or to approximately 90° F., or from 80° F. or from approximately 80° F. to 90° F. or to approximately 90° F., or to and from any values within these ranges.

Any embodiments disclosed herein can also be configured to use one or more substances that polymerize with activation by any number of various wavelengths of light, or are activated by light. Activation light may be provided by special applicator within or separate from the material delivery device. Suitable substances that can be used also include substances that are typically used in or are approved for use in subretinal space, a vitreous cavity, or an ocular surface including on or under conjunctiva, and substances that may be used for closing gaps in tissue.

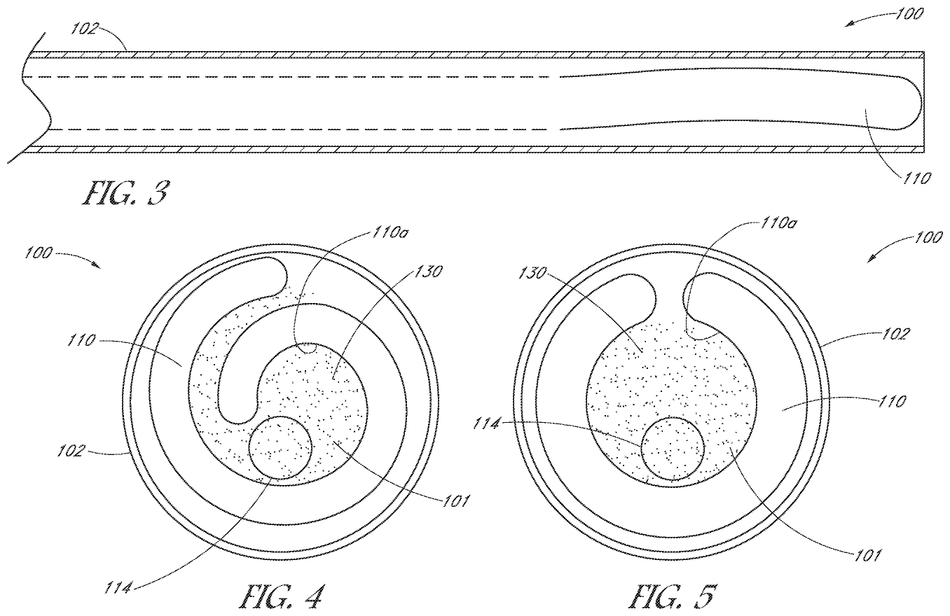

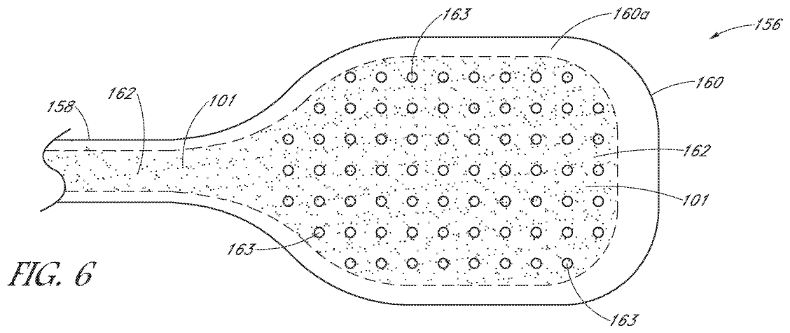

In any embodiments, the applicator portion 110 can be biased to be sufficiently flexible or otherwise structurally biased to be collapsible in the width direction W so as to be withdrawable within the outer sleeve 102 and also so as to minimize forces exerted on and resultant trauma that may occur from a more rigid material. FIGS. 3 and 4 depict a side view and an end view, respectively, of the embodiment of the device 100 shown in FIG. 1, showing the device in a first or withdrawn state wherein the applicator portion 110 is contained or withdrawn into the outer sleeve 102. FIG. 5 is an end view of another embodiment of the device 100 and the applicator portion 110.

The inner sleeve 104 and applicator 106 can optionally be advanceable and withdrawable through the lumen or passageway 103 of the outer sleeve 102, such as between the first state illustrated in FIGS. 3-4 and the second or deployed state illustrated in FIGS. 1-2.

When the applicator 106 is in the first or withdrawn state, the distal portion of the device 100 can have a smaller profile or cross-sectional size which can be defined by the outer sleeve 102 so as to be advanceable through an opening in a tissue surface with less force required and, consequently, less risk of trauma to the tissue. Some embodiments of the device 100 can be configured such that, when the applicator portion 110 is withdrawn within or position within the outer sleeve 102, the applicator portion 110 can take a curved or helical shape so as to fit within the inner space of the outer sleeve 102, as best shown in FIGS. 4 and 5 and, thereby, form a space 130 confined by the first surface 110a of the applicator portion 110.

With reference to FIG. 5, the applicator portion 110 can be configured such that, when the inner sleeve 104 and the applicator 106 all withdrawn back into the outer sleeve 102 from the second state to the first or withdrawn state, the applicator portion 110 can be biased or otherwise configured to collapse to a more compact or narrow shape or state described above and shown in FIG. 5 so that the device 100 can be withdrawn through the opening in the tissue with the applicator 106 withdrawn with in the outer sleeve 102. As shown and described, the applicator portion 110 can have a width W that is greater than a width of the outer sleeve 102. Because the applicator portion 110 is configured or biased to move to a more compact profile, withdrawing the applicator portion 110 within the outer sleeve 102 as shown in FIGS. 4 and 5 can reduce the size of the distal portion of the device 100 to the size of the outer sleeve 102. This can reduce the trauma to the tissue when the device 100 is withdrawn through any openings, if any, in the tissue. In any embodiments, the applicator portion 110 can be configured to have any features or be made from any materials that allow it to be folded, rolled, compressed, squeezed, narrowed or otherwise reduced in width W or size to fit within the outer sleeve.

The applicator portion 110 can have or define a first width when the applicator portion 110 is in the first position (wherein the applicator portion 110 is contained within the outer sleeve 102) and a second width when the applicator portion 110 is the second position (wherein the applicator tip extends past the opening at the distal end 102a of the outer sleeve 102). The second width of the applicator portion 110 can be substantially greater than the first width of the applicator portion 110. In any embodiments disclosed herein, the second width of the applicator portion 110 can be approximately 5 times greater than the first width of the applicator portion 110, or from 2 times or approximately 2 times greater (or, optionally, less) to 8 times or approximately 8 times greater (or, optionally, more) than the first width of the applicator portion 110, or from 3 times or approximately 3 times greater to 6 times or approximately 6 times greater than the first width of the applicator portion 110.

In any embodiments disclosed herein, the second width of the applicator portion 110 can be 2 mm, or approximately 2 mm, or 3 mm, or approximately 3 mm, or 4 mm, or approximately 4 mm, or from 1.5 mm or approximately 1.5 mm (or smaller) to 6 mm or approximately 6 mm (or larger), or from 2 mm or approximately 2 mm to 4 mm or approximately 4 mm, or from 2 mm or approximately 2 mm to 3 mm or approximately 3 mm, or any sizes within these ranges or from and to any sizes within these ranges. In other embodiments, wherein the device may be used for non-ocular tissue, the second width can be any values or from and to any values that are 2 times greater than the above-listed values or ranges, or 3 times greater than the above-listed values or ranges.

The applicator portion 110 can be made of a solid, non-fenestrated material. Or, alternatively, the applicator portion 110 can be made of a fenestrated material that has many openings or slits, or any combination thereof, like a sieve or fly swatter. The openings or slits can optionally be of variable sizes and/or dimensions.

An additional benefit of withdrawing the applicator portion 110 within the sleeve 102 is that, when the substance 101 is advanced through the passageways formed in the inner sleeve 104 and the applicator 106, the substance 101 can fill the space 130 and thereby come into contact with and substantially cover the first surface 110a of the applicator portion 110. In this embodiment, to improve the coverage of the substance 101 on the first surface 110a of the applicator portion 110, the user can withdraw the applicator portion 110 within the outer sleeve so as to form the circular or rolled shape of the applicator portion 110 and create the space 130 within which the substance 101 can be advanced and, optionally, substantially filled.

In any embodiments, the applicator portion 110 can be withdrawn within the sleeve 102 in an initial state of the device 100. The substance 101 can optionally be pre-dispensed within the passageways 105, 112 and within the volume 130 before the device 100 is advanced into the target location. Additionally, the substance can optionally be stored within the passageways 105, 112 and within the volume 130 before the device is put into use. A thermoresponsive gel or other heat activated substance can be used in such embodiments to increase the shelf-life of a pre-filled device and reduce the likelihood of the substance curing during storage of the device or before application of the device. Heat from the patient's body or an added heat source (for example and without limitation, a heat source integrated into or applied to the applicator portion 110) can be used to activate the thermoresponsive substance. In other embodiments, the user or surgeon can dispense the substance 101 through the passageways 105, 112 after the device 100 is advanced to the target location and/or after the applicator portion 100 is advanced to the second or deployed state or position (shown in FIG. 1).

The device 100 can optionally be configured such that the applicator 106 can be selectively connectable to the inner sleeve 104 at the connection 122. In this configuration, the device 100 can enable multiple additional capabilities that are not possible without the connectable interface 122. As a first example, after the desired procedures have been formed in the substance 101 has been applied to the target surface, a user of the device can disconnect the applicator 106 from the inner sleeve 104 at the connector 122 so that outer sleeve 102 and inner sleeve 104 can be withdrawn from the target location without also withdrawing the applicator 106 or portion thereof, such as the applicator portion 110. For example, in some applications such as ocular applications, it may be desirable to leave the applicator 106 in contact with the ocular tissue until the desired level of healing has been achieved or indefinitely. Thereafter, the distal tip 106 or portion thereof can remain in the target location or can be removed from the target location by any suitable means, including by reconnecting the applicator 106 with the inner sleeve 104 and withdrawing the applicator 106 within the outer sleeve 102. The distal tip 106 can be made from a bioabsorbable material, an inert material, or otherwise. If inert and left in the target location, the applicator portion 110 can provide additional scaffolding or support to the tissue, even after the substance has dissolved or dissipated.

In any embodiments, any portions of the device 100, including without limitation the passageway 105 of the inner sleeve 104, can be prefilled with a substance 101 and stored in this partially filled condition. The applicator 106 can be provided in an unattached or disconnected state wherein the applicator 106 is disconnected from the inner sleeve 104 and is free of any substance 101. In this configuration, after the applicator 106 is coupled with the inner sleeve 104 at the connector 122, the user can advance the substance 101 through the passageway 112 of the elongated portion 108 of the application tip 106, out through the opening 114 and onto the applicator portion 110 when the applicator portion 110 is either the first state or the second state.

The connector 122 in any embodiments disclosed herein can be configured to be self-sealing such that, when the applicator 106 is disconnected from the inner sleeve 104, the connector 122 can seal the passageway 105 of the inner sleeve 104 to prevent the expulsion of any substance within the passageway number 105 of the inner sleeve 104 through the connector 122.

Any embodiments of the applicator portion 110 can optionally be configured to be less flexible in the lengthwise direction (indicated by L in FIG. 2) than in the width direction W. The material of the applicator portion 110 can be configured to have stiffening features or materials, such as without limitation, corrugations, stiffening wires, wire loops, ribs, sheets, or other structural members made out of Nitinol, stainless steel, or other alloys, channels, ribs, or other features such as in a lengthwise direction to increase a stiffness of the applicator portion 110 in the lengthwise direction and decrease a stiffness of the applicator portion 110 in the widthwise direction.

Additionally, some embodiments can optionally be configured to dispense or apply a flexible membrane of the substance to a surface, or to deliver any substance or material to an enclosed space, such as an eye, through a small opening in the eye.

FIG. 6 depicts a top view of another embodiment of an applicator tip 156 that can be used with any of the embodiments of the devices disclosed herein, including without limitation any of the devices 100 disclosed above. The applicator tip 156 can include any of the features, materials, or other details of any of the other applicator tip embodiments disclosed herein, including without limitation applicator 106 described above, in combination with or in place of any of the features described below to form new embodiments.

With reference to FIG. 6, the applicator portion 160 can include a plurality of openings 163 in fluid communication with the passageway 162 extending through the elongate portion 158. The plurality of openings 163 can be formed about the first surface 160a of the applicator portion 160 in any desired pattern and in any desired number. For example and without limitation, the applicator portion 160 can have approximately 20 openings 163 formed therein, or from 5 or approximately 5 to 40 or approximately 40 openings formed therein, or from 10 or approximately 10 to 30 or approximately 30 openings formed therein, or from 15 or approximately 15 to 25 or approximately 25 openings formed therein, or of approximately any number of openings 163 within these ranges, or from and to any number of openings 163 within these ranges.

The openings 163 can provide for or enable a more even distribution of substance 101 across the first surface 160a of the applicator portion 160. Additionally, the openings 163, because they extend only through the first surface 160a of the applicator portion 160 to the passageway 162, can increase the flexibility and/or collapsibility of the applicator portion 160 so that the applicator portion 160 to facilitate the collapse, bending, and/or withdrawal of the applicator portion 160 within the outer sleeve of the device when the device is in the first or withdrawn state.

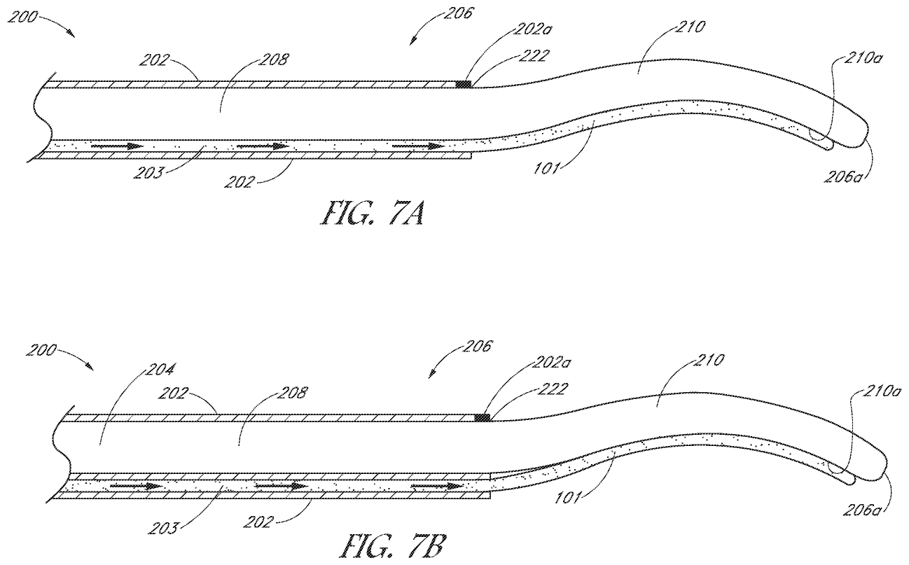

FIG. 7A depicts a section view of another embodiment of a device 200 for application of a substance 101 to a surface of an object, showing the device 200 in the second or deployed state. The device 200 can include any of the features, materials, or other details of any of the other device embodiments disclosed herein, including without limitation device 100 described above, in combination with or in place of any of the features described below.

In some embodiments, the device 200 can include a cannula or outer sleeve 202 (also referred to herein as an elongate body) having a lumen or passageway 203 therein through which the substance 101 can be advanced, and an applicator tip 206 coupled with an inner core 204. The applicator tip 206 and the inner core 204 can be movable (e.g., extendable and retractable) within the outer sleeve 202. In some embodiments, the applicator tip 206 and the inner core 204 can be rotatable within the outer sleeve 202. The outer sleeve 202 can be configured to provide additional rigidity and manipulability to the applicator tip 206 to facilitate or improve the user's control of the device 200 during application of the substance 101, and also to advance the substance through the device 200 to an applicator portion 210 of the applicator tip 206.

A distal end portion 206a of the applicator tip 206 can extend out of a distal end 202a of the cannula or outer sleeve 202. In any embodiments, the applicator tip 206 can have an elongate portion 208 and an applicator portion 210. A seal 222 can optionally be positioned at a distal end 202a of the outer sleeve 202 of the device 200 to seal around an outside surface of the elongate body 208 except for a channel or passageway that is aligned with a first surface 210a (which can be referred to as an applicator surface) of the applicator portion 210. Additionally, as with any embodiments disclosed herein, fluoroscopic or radiopaque markers can be positioned along the outer sheath 202, such as a distal end 202a of the outer sheath 202, and/or along the applicator tip 206 to provide visualization to a surgeon of the location of these components.

With this embodiment, as shown in FIG. 7A, a separate lumen through the elongate body 208 and applicator tip 206 need not be formed since the substance 101 can be communicated through the space inside the outer sleeve 202 between the outside surface of the elongate body 208 and the inside surface of the outer sleeve 202. In some embodiments, a channel or space can be formed along a length of an outside surface of the inner core 204 or an inside surface of the outer sleeve 202 along which or through which the substance 101 can be advanced. In other embodiments, as shown in FIG. 7B, the outer sleeve 202 can have a separate lumen 203 through which the substance 101 can be advanced.

In some embodiments, the elongate body 208 can extend from at least a proximal end of the outer sleeve 202 to the applicator portion 210, such that no separate inner core is needed. In some embodiments, the proximal end of the elongate body 208 or the inner core 204 can extend past or through the handle (optionally, with appropriate seals, as needed) so that the user can manipulate the elongate body 208 to advance and withdraw the applicator portion 210 as needed. Similar to applicator portion 110 described above, the applicator portion 210 can be configured to be withdrawable within the outer sleeve 202 and be configured to be moveable between the first or closed state and the second or extended state. For example and without limitation, the applicator tip 206 can be withdrawn into the outer sleeve 202 and then extended as substance 101 is advanced past a distal end 202a of the outer sleeve 202, thereby applying the substance 101 to a distal portion 206a of the applicator tip 206 and along a length of the applicator tip 206 as the applicator tip 206 is advanced distally relative to the distal end 202a of the outer sleeve 202.

In any embodiments of the device 200, the handle, cannula or outer sheath 202, the inner sheath 204, or other rigid or semi-rigid components can be made from or comprise a any suitable plastic material, metal, polyvinyl chloride, glass, acrylic, carbon fibers and/or any combination of the foregoing. In some embodiments, the cannula tip plug or protective tip cover can comprise plastic, metal, polyvinyl chloride, glass, acrylic, carbon fibers, rubber (such as, without limitation, silicon), and/or any combination of the foregoing.

Any embodiments disclosed herein can have a handle (not shown) at the proximal end of the device 100. The handle can have any details of any contemporary or existing designs of biomaterial or other liquid material delivery devices. In some embodiments, the handle can have a first handle portion or member that can be coupled to the outer sleeve 102 of the device 100. An additional or second handle can be coupled with the inner sleeve 104. The second handle member can be independently movable relative to the first handle member so that a user can advance and withdraw the inner sleeve 104 and, consequently, the applicator 106, if coupled with the inner sleeve 104, relative to the outer sleeve 102.

A dispensing device or dispensing means (not illustrated) for dispensing the substance 101 into the passageway 105 can be in fluid communication with a proximal end 104b of the inner sleeve 104. The dispensing device can be coupled with or otherwise part of the handle of the device 100. In some embodiments, the dispensing device can include a plunger or syringe, a fillable chamber that can be filled or contain the substance, a compressible bulb, a compressible bladder, or other device configured to eject the substance 101 through the passageway 105. Additional mechanisms for advancing the substance 101 into the inner sleeve 104 can include a mechanical pump, a powered pump, a pneumatic pump or cylinder, an electromechanical pumping means, etc. Optionally, the handle can include a port configured to receive an adhesive material into the handle. A bladder of material or other ejector device, such as is described herein, can be coupled in fluid communication with the port.

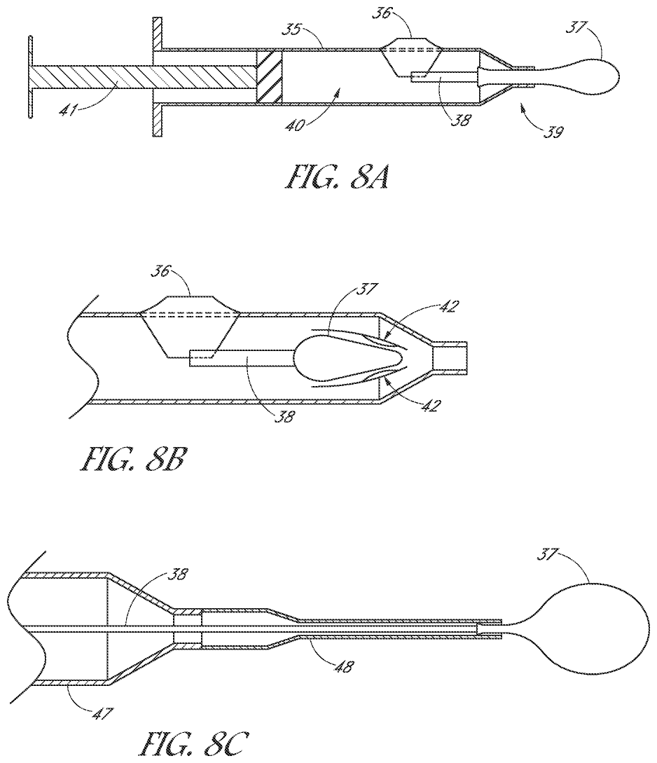

FIG. 8A depicts an example of a system and/or device including a plunger 41, a handle 35, a cavity 40 for holding a bioadhesive material, an extender knob 36, a rod 38, a locking connector 39, and a flexible loop 37 containing a film of bioadhesive material 37 (partially extended). The plunger 41 may be used to expel a bioadhesive material through a hollow cannula and/or into the flexible loop 37 when pressed on by a user (for example, pressure from a user's finger or using pneumatic pressure supplied by an external device connected to the handle). In some embodiments, the plunger 41 is incorporated into the examples in FIGS. 8B and/or 8C. For example, the extender knob 36 may be used to extend the flexible loop 37 from a retracted position as seen in FIG. 8B. In some embodiments, the flexible loop 37 can be substituted with any of the applicator tips as disclosed elsewhere herein.

FIG. 8B depicts an example of a system and/or device that includes an extender knob 36, a rod 38, a flexible loop containing a film of bioadhesive material 37 and two guide ridges 42. Some embodiments include a retractable loop such as in FIG. 8B that expands out as shown in FIG. 8A and/or FIG. 15C.

FIG. 8C depicts an example of a system and/or device that includes a handle 47, a rod 38, a cannula 48, and a flexible loop containing a film of bioadhesive material 37. The cannula may be detachable or non-detachable.

Any components of the device 100 in any embodiments disclosed herein, including without limitation the handle portion, the outer sleeve 102, the inner sleeve 104, and/or the distal tip 106 can be configured to have temperature modulating capabilities. In some embodiments, the outer sleeve 102, the inner sleeve 104, and/or the distal tip 106 can optionally be configured to dispel heat from the device that may result from any heat generated by the substance 101 as the substance 101 is advanced through the device 100. For example, one or more of the outer sleeve 102, the inner sleeve 104, and the distal tip 106 can optionally be made from materials or contain other materials such as thermally conductive metals (like copper, steel, Nitinol, aluminum, or other alloys, etc.) that have improved or enhanced thermal conduction which can conduct heat away from certain portions of the device 100. In some embodiments, the outer sleeve 102 and/or the inner sleeve 104 can optionally be configured to conduct or dispel heat, while the applicator 106 is not configured to dispel or conduct heat.

Alternatively, the outer sleeve 102, the inner sleeve 104, and/or the distal tip 106 can optionally be configured to retain heat within one or more components of the device that may result from any heat generated by the substance 101 as the substance 101 is advanced through the device 100. For example, one or more of the outer sleeve 102, the inner sleeve 104, and the distal tip 106 can optionally be made from materials that have improved or enhanced thermal insulating capabilities which can reduce the amount of heat conducted away from certain portions of the device 100. In some embodiments, the outer sleeve 102 and/or the inner sleeve 104 can optionally be configured to retain heat, while the applicator 106 is not configured to retain heat.

In some embodiments, one or more of the components of the device 100 (which can include the outer sleeve 102 and the inner sleeve 104, or, optionally, the outer sleeve 102, the inner sleeve 104, and the applicator 106) can be pre-cooled to a desired temperature that is lower than the room temperature or the body temperature such that, if any heat is generated by the substance 101, the temperature of the device 100 will not elevate above a desired temperature threshold which, optionally, can be the body temperature of the patient.

Additionally, in some embodiments, one or more of the components of the device 100 (which can include the outer sleeve 102 and the inner sleeve 104, or, optionally, the outer sleeve 102, the inner sleeve 104, and the applicator 106) can be configured to maintain the substance 101 (which can be a hydrogel) at a temperature that is below a threshold temperature value so that the adhesive properties or capabilities of the substance are reduced when the substance 101 is contained within the device. Upon advancement of the substance 101 onto the applicator portion 110 or application of the substance 101 on the target tissue or surface, the temperature of the substance and, consequently, the adhesive properties, can increase. For example and without limitation, the outer sleeve 102 can be sized and configured such that an inner diameter of the passageway 103 is greater than an outside surface of the inner sleeve 104 so as to create a space between the inside surface of the outer sleeve 102 and the outside surface of the inner sleeve 104. A cooling fluid can be channeled through individual lumen or otherwise through the passageway 103 to provide a source of cooling to the inner sleeve 104 and, consequently, the substance 101. One or more seals can be used to seal an end of the outer sleeve 102 to the inner sleeve 104 so that none of the cooling fluid is leaked from the device 101. Alternatively, an outer shell that is configured to surround the outer sleeve 102 can be positioned about the entire outer sleeve 102 to provide an additional cavity or passageway for communication of a cooling fluid that will reduce the temperature within the outer sleeve 102 and, consequently, of the substance 101. An inlet port and an outlet port can be formed in or coupled with the handle for the inlet and outlet of cooling fluid through the device 100. The cooling fluid can optionally include ammonium nitrate.

Optionally, a rheostat and/or other means can be positioned within the handle and can be used to control the temperature of any components of or all of the device 100. Optionally, one or more thermal sensors or other sensors can be used to monitor the temperature of any components of or all of the device 100. Additionally, any embodiments of the device 100 disclosed herein can be configured to have the capability to automatically maintain a temperature of portions of the device within a specified range, even when the substance or application may tend to increase the temperature of the device or components thereof.

Additionally, any embodiments of the device 100 disclosed herein can be configured to transfer heat to one or more of the components of the device 100 (which can include the outer sleeve 102 and the inner sleeve 104, or, optionally, the outer sleeve 102, the inner sleeve 104, and the applicator 106, or, optionally, just the applicator 106 or applicator portion 110) to elevate a temperature of the substance 101 contained within the device and/or the applicator tip to transfer or deliver heat to the target tissue. In some embodiments, thermally conducting materials such as, without limitation, copper, steel, Nitinol, aluminum, or other alloys, etc., can be integrated into the device and extend lengthwise along the device to transfer heat from a heat source adjacent to a proximal end or handle of the device to the applicator 106. Optionally, one or more thermoelectric components or means can be used to transfer heat to one or more of the components of the device 100 (which can include the outer sleeve 102 and the inner sleeve 104, or, optionally, the outer sleeve 102, the inner sleeve 104, and the applicator 106, or, optionally, just the applicator 106 or applicator portion 110).

In any embodiments disclosed herein, the device or method can include removing or liquefying solidified substance or gel from the device 100 or tissue surface (such as the tissue on the surface of the eye), if desirable, by increasing or decreasing a temperature of the inner sleeve 104, applicator 106 or applicator portion 110, depending on the specific thermoresponsive properties of the material. The liquefied substance or gel can then be aspirated through the device and removed through the proximal end of the device or handle portion. The device can optionally be configured to have one or more aspiration tubes or channels to aspirate any liquefied substance or gel, or any other substance that is desired to be removed from the device. The tubes can optionally be heated or temperature controlled to ensure that the removed substance remains in a liquid state.

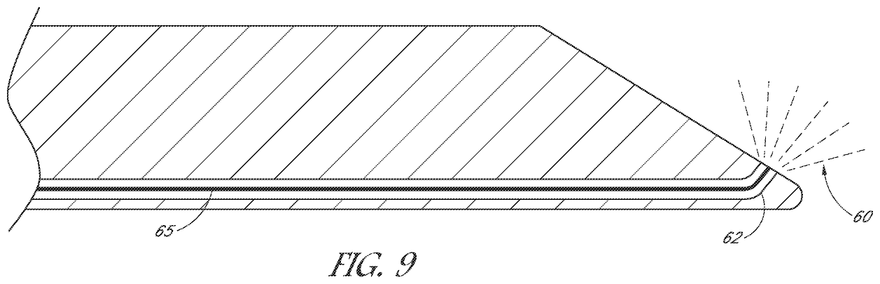

Any embodiments of the devices disclosed herein can include an internal cautery component. Such embodiments can include a cannula with a fiber or wire for light or cautery. FIG. 9 depicts an example of a cannula with a fiber or wire for light or cautery. The example includes a light fiber 65 within a tube along an edge of the cannula. The light fiber 65 includes an angled end 62 in the example, but in some embodiments the end of the light fiber 65 is straight. The end 62 of the light fiber 65 can be beveled at an angle that matches the angle of the bevel of the cannula tip, if so angled. The example includes an illumination 60 emitted by the end 62 of the light fiber 65.

In some embodiments, the cannula tip can be configured to be used for cautery. For example, an extra tube can be included that is configured for passage of an additional material (such as a liquid) that can be expressed from the cannula tip as a spray or as drops. For example, the additional material may be used to activate the bioadhesive material. In some embodiments, the device or system, or a component thereof such as the handle, cannula, cannula tip, light, or light fiber can be configured to emit a light at a wavelength that activates, thickens, or hardens the bioadhesive material.

The system and/or device can be activated in a manner that increases the temperature at the cannula tip (using, for example, the internal cautery component). This is advantageous according to some embodiments in aiding disengagement of the bioadhesive from the applicator portions in instances where the bioadhesive material is a temperature-responsive material that becomes more fluid at higher temperatures. Some embodiments include a cryotherapy component such as a cryotherapy handpiece. In some embodiments, the system or device may be activated in a manner that decreases the temperature at the applicator tip (using, for example, a cryotherapy component). This is advantageous according to some embodiments in aiding disengagement of the bioadhesive from the applicator portion in instances where the bioadhesive material is a temperature-responsive material that becomes more fluid, friable, or releasable at lower temperatures.

The substance used with any of the embodiments disclosed herein can include a thermally activated substance. Additionally, the device can optionally be configured to provide a source of warm air or cold air onto the substance through an additional lumen within the inner cannula 104, through an additional lumen or passageway outside of the inner cannula 104, through a completely separate cannula from a separate device, or otherwise. The warm air or cold air can be blown onto the applied substance. The device can optionally include a filter to filter the air before warming or cooling it and applying it to the substance. In any embodiments, the air source passageway can have any of the sizes disclosed herein for the outer cannula 102 and/or the inner cannula 104. The source of warm or cold air can be a free-standing piece of equipment, can be integrated into the outer or inner cannula, can be integrated into other surgical devices such as a vitrectomy machine that is configured to filter the air and pass it to the eye or other instruments.

Any embodiments of the devices disclosed herein can be incorporated or included in a kit or a system, also comprising one or more substances (which can be any of the substances disclosed herein or other substances now known or later developed). The kit can also include a material dispenser for dispensing the substance, if not included with the device. The kit can also optionally include a tip plug or protective cover for a distal end of the device, if not already included with the device. The system, device, or kit can be sterile or pre-sterilized. In some embodiments, the kit includes a pre-sterilized material dispenser as described herein.

The kit or system can also include any of the other components or devices disclosed herein, including, for example and without limitation, one or more of the curing components and devices for applying the curing components to the substance after the substance has been applied to the target tissue surface. Additionally, in any embodiments disclosed herein, the substance or one or more different substances may be provided in a capsule or a self-contained syringe that can be coupled with an opening, port, or otherwise in the handle portion of the device.

Some embodiments relate to a use of a system, device or kit for applying a substance (which can comprise any of the substances listed above, including a bioadhesive) to an eye or retina such as for repair of the eye including the retina, sclera, conjunctiva, and cornea, etc. In any such methods, the cannula can optionally be advanced into the eye using any known or commonly used techniques. The surgical procedures disclosed herein can include piercing the surface of the eye or other tissue of the eye, or can include treating the eye tissue without entering or piercing any eye tissue.

The methods disclosed herein can include advancing the substance to the applicator portion of the applicator tip and from the applicator portion to the target tissue, which can be eye tissue or, optionally, retinal tissue. Any embodiments disclosed herein can optionally be used to repair a retinal tear or other defects or tissues within the body, as disclosed elsewhere herein and other surgical applications beyond what is disclosed herein. Some embodiments include operation of a button on the handpiece of the device or system, other manual controls, or operation of a foot pedal to, for example, control a valve to control aspiration of the substance, infusion of the substance, infusion or delivery of an activation solution or other curing means, allow the substance to flow through the passageway of the handpiece and/or through the flow passageway(s) of the devices, etc.

Any embodiments disclosed herein can include a means for curing the substance after it has been applied to the target surface, including light emitting equipment, UV emitting equipment, application of increased temperature (e.g., applying heat), application of reduced temperature (e.g., applying cooling), or otherwise.

Any embodiments disclosed herein can also be configured for applications outside of the eye tissue, including without limitation for applications in gastrointestinal tissue, colon tissue, etc.

In some embodiments, the material delivered by the device can be delivered transvitreally. For transvitreal use, delivery through the device may employ a cutting action to remove the cohesive/sticky material once it is delivered. In some embodiments, excess is be removable with a vitrectomy cutter and/or aspiration. Transvitreal delivery may be utilized in air filled eye to avoid adhesive material floating to non-targeted ocular structures such as lens/IOL/pars plicata/angle, corneal endothelieum which could result in obscuration of view through lens/IOL or impairment of inflow/outflow of aqueous humor.

During application of the substance, one or more portions of the tissue can be covered with a fluid to protect such tissues or mask such tissue to avoid contact from the substance. Additionally, air can be used to control an application of the substance and/or avoid contact of some tissue surfaces by the substance. For example and without limitation, an air disc can be created to cover or protect a tissue surface, wherein the adhesive material can settle on a fluid surface rather than on underlying structures, and/or be immediately activated (made non-sticky) to avoid adhesion to non-target tissues. Activation in any embodiments disclosed herein can include activation by water or similar liquid. If the substance is thermoreactive, any undesirable material can have its temperature modulated by any embodiments of this device so the material can be converted to liquid form, after which such material can be aspirated from the eye.

Trans-scleral delivery is contemplated, and may include delivery of material to the retinal break through the subretinal space. After the material sticks to the retina after being delivered via a tapered end of the device, or needle, the material may be amputated as the needle is withdrawn from the sclera. The material may act as a buffer to prevent retinal incarceration into sclera defect left by the cannula, needle, or device utilized to cross the scleral and choroidal tissue.

The delivery device may be single, double, or triple bored. In some embodiments, the double bore device includes one bore for delivery of a material and a second bore for reflux of intraocular fluid to outside the eye as TOP increases. In some embodiments, one of the bores contains a light fiber that may be used for illumination. In some embodiments, one of the bores contains a fiber that delivers a laser light. In some embodiments, one of the bores contains a fiber that delivers heat or some other source of energy used for curing the injected material.

In some embodiments, the triple bore device includes one bore for delivery of a material, one bore for passage of gas used to create bubble or film of adhesive material, and a third bore for reflux of intraocular fluid to outside the eye as IOP increases.

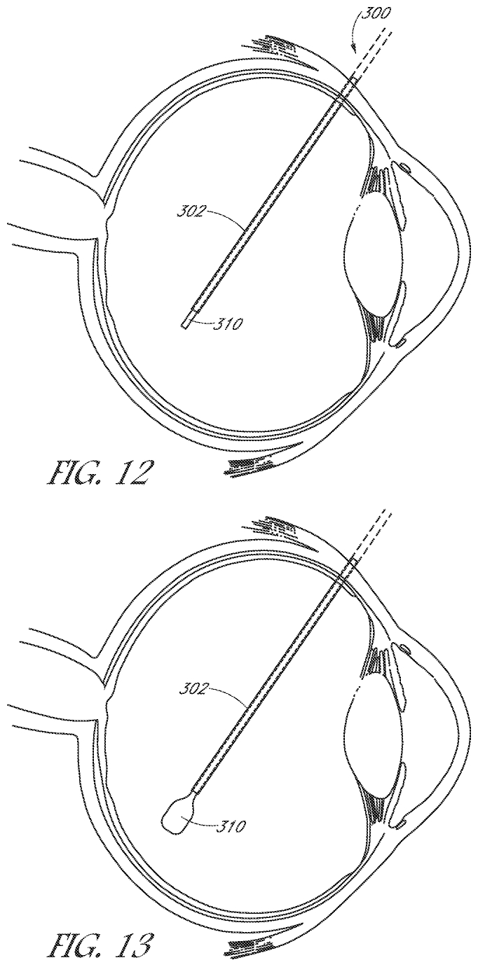

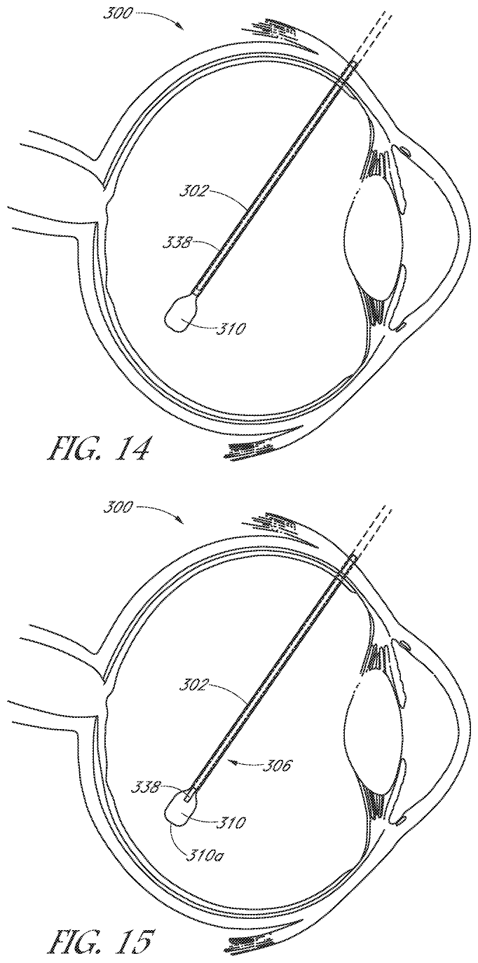

FIGS. 12-17 illustrate one optional method of using the embodiment of the device 300. With reference to FIG. 12, a cannula 302 can be advanced into the eye. An applicator portion 310 can be positioned within or withdrawn within the cannula 302 as the cannula is advanced into the eye, as shown in FIG. 12. In some embodiments, the applicator portion 310 can have a substance on the applicator portion 310 when the applicator portion is positioned within the cannula 302. In some embodiments, as discussed below, the substance can be applied to the applicator portion 310 after the applicator portion 310 is advanced distally past a distal end of the cannula 302. Thereafter, as shown in FIG. 13, the applicator portion 310 can be extended from the distal end of the cannula 302 and permitted to expand to the second or expanded state. The cannula 338 (also referred to herein as a supply cannula) can be extended through the outer cannula 302. FIG. 14 shows the cannula 338 extended so that a distal end of the cannula 338 is approximately aligned with a distal end of the outer cannula 302. The cannula 338 can be extended past the distal end of the outer cannula 302 toward a distal end 310a of the applicator portion 310 of the applicator tip, as shown in FIG. 15. Thereafter, as shown in FIG. 16, a substance 101 can be advanced through the passageway 340 of the cannula 338 and ejected onto the applicator portion 101. Thereafter, the outer cannula 302 and the applicator portion 310 can be moved and manipulated by the user to apply the substance 101 to the target tissue, as shown in FIG. 17.

Some embodiments of the system and/or device further include an internal material supply chamber within the handpiece or cannula, for cooling or heating fluids, or other fluids or gases that can be used with the device 100. The internal material supply chamber can optionally include an elongate body comprising a proximal end and a distal end comprising a material supply tip and/or exit port. The internal material supply chamber can optionally be internal to or adjacent to at least a portion of the passageway 105.

In some embodiments of the system and/or device, the internal material supply chamber, elongate body of the internal material supply chamber, material supply tip and/or material supply exit port comprises a 0.1-0.5 mm, 0.5-1 mm, 1-2 mm, 2-3 mm, 3-4 mm, 4-5 mm, 5-6 mm, 6-7 mm, 7-8 mm, 8-9 mm, 9-10 mm, 10-20 mm, 20-30 mm, 30-40 mm or 40-50 mm cross-sectional diameter. In some embodiments of the system or device, the internal material supply chamber, elongate body of the internal material supply chamber, material supply tip and/or material supply exit port comprises a length of 0.1-0.5 mm, 0.5-1 mm, 1-2 mm, 2-3 mm, 3-4 mm, 4-5 mm, 5-6 mm, 6-7 mm, 7-8 mm, 8-9 mm, 9-10 mm, 10-20 mm, 20-30 mm, 30-40 mm, 40-50 mm, 50-60 mm, 60-70 mm, 70-80 mm, 80-90 mm, 90-100 mm, 100-150 mm or 150-200 mm.

Some embodiments of the system and/or device further comprise a second internal material supply chamber. In some embodiments of the system or device, the internal material supply chamber and the second internal material supply chamber each comprises a separate bioadhesive material, for example a polyethylene glycol solution and a trilysine amine solution, hydrogel, double layer hydrogel, or polymeric hydrogel.

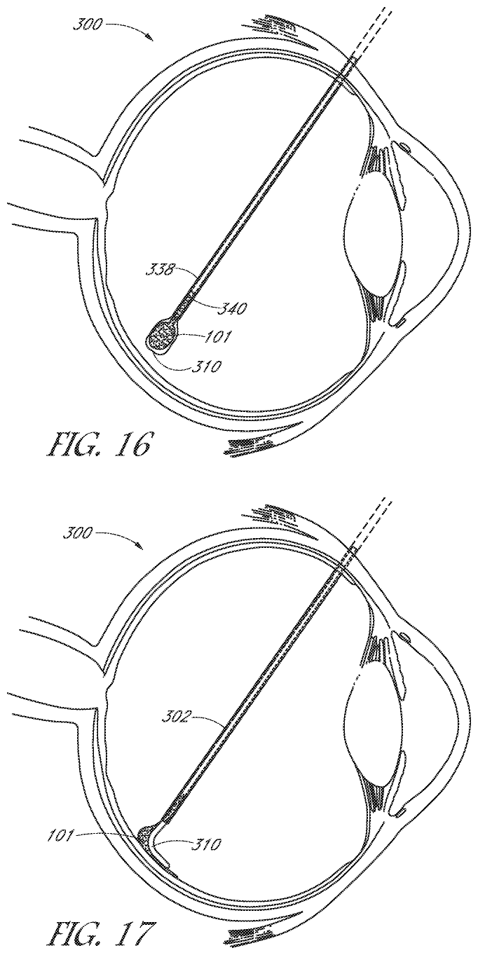

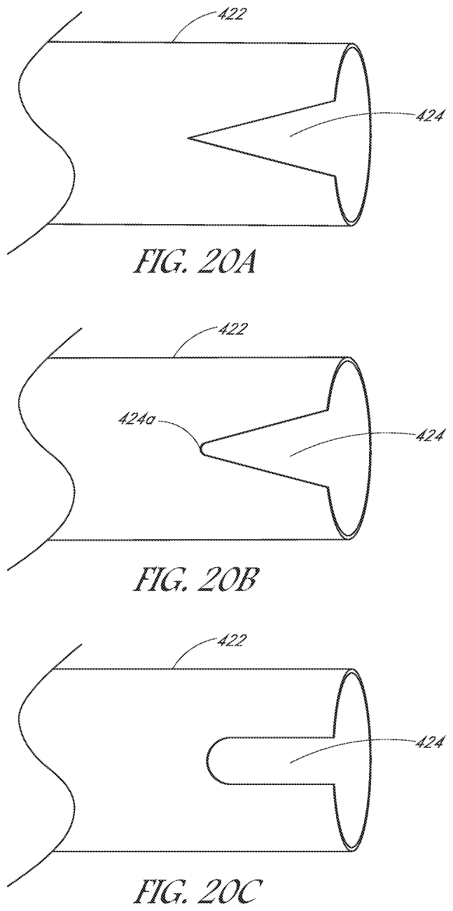

FIG. 18A is a perspective view of an end portion of an embodiment of a cannula 402 that can be used with any embodiments of the devices disclosed herein. FIG. 18B is a section view of the embodiment of the cannula 402 shown in FIG. 18A, taken through line 18B-18B of FIG. 18A. FIG. 18C is a top view of the embodiment of the cannula 402 shown in FIG. 18A. As shown, the cannula 402 can have a notch or cutout 404 formed in an end portion of the cannula 402 which can assist in the retraction or reduction in size (e.g., the constriction or collapse) of any of the applicator portion embodiments disclosed herein as the applicator portion is retracted into the distal end of the outer sleeve or cannula. With reference to FIG. 18C, in some embodiments, the cutout 404 can have an asymmetric shape about a plane extending through an axial centerline C of the cannula 402—for example and without limitation, through an plane that is through the axial centerline C and is perpendicular to the plane of the figure (e.g., perpendicular to the plane of the paper). In some embodiments, the notch 404 can terminate in a curved end portion 406. In other embodiments, the notch 404 can have a triangular shape wherein one side of the notch is parallel to the axial centerline of the cannula. In other embodiments, the notch can have a triangular shape wherein both sides of the notch are tapered or angled or are otherwise in a “v” shape, such as in the notch 424 of the cannula 422 shown in FIGS. 20A and 20B. In some embodiments, as shown in FIG. 20B, the apex or proximal end portion 424a of the v shaped notch can be rounded or have a radius. In other embodiments, the notch can have a rounded end portion and parallel side portions, or otherwise have a generally “u” shape, as in FIG. 20C.

Additionally, with reference to FIG. 18C, the notch in any embodiments disclosed herein (including without limitation the notches 404, 414, and 424) can have a length L and a width W adjacent to the end of the cannula 402, and the embodiments of the cannulas disclosed herein can have a diameter D. For example and without limitation, the width W of the notch (or width W1 for the notch 414) can be 30% (or approximately 30%) of the diameter D of the cannula, or from 20% (or approximately 20%, or less than 20%) to 100% (or approximately 100%, or more than 100%) of the diameter D of the cannula, or from 30% (or from approximately 30%) to 50% (or approximately 50%) of the diameter D of the cannula, or of any value or from and to any values within these ranges. In some embodiments, the width W of the notch (or width W1 for the notch 414) can be 0.25 mm (or approximately 0.25 mm), or from 0.1 mm (or, in some embodiments, approximately 0.1 mm or less than 0.1 mm) to 1.5 mm (or, in some embodiments, approximately 1.5 mm or more than 1.5 mm), or from 0.25 mm (or, in some embodiments, approximately 0.25 mm) to 0.75 mm (or, in some embodiments, approximately 0.75 mm), or of any value or from and to any values within these ranges.

In any embodiments, the length L of the notch of any embodiments disclosed herein can be approximately 30% of a length of the applicator portion, or from 20% (or approximately 20% or less than 20%) to 100% (or approximately 100% or more than 100%) of the length of the applicator portion, or from 30% (or approximately 30%) to 60% (or approximately 60%) of the length of the applicator portion, or from 30% (or approximately 30%) to 50% (or approximately 50%) of the length of the applicator portion, or of any value or from and to any values within these ranges. In some embodiments, the length of the notch can be approximately 1 mm, or from 0.5 mm (or, in some embodiments, approximately 0.5 mm or less than 0.5 mm) to 2 mm (or, in some embodiments, approximately 2 mm or more than 2 mm), or from 0.75 mm (or, in some embodiments, approximately 0.75 mm) to 1 mm (or, in some embodiments, approximately 1 mm), or of any value or from and to any values within these ranges.

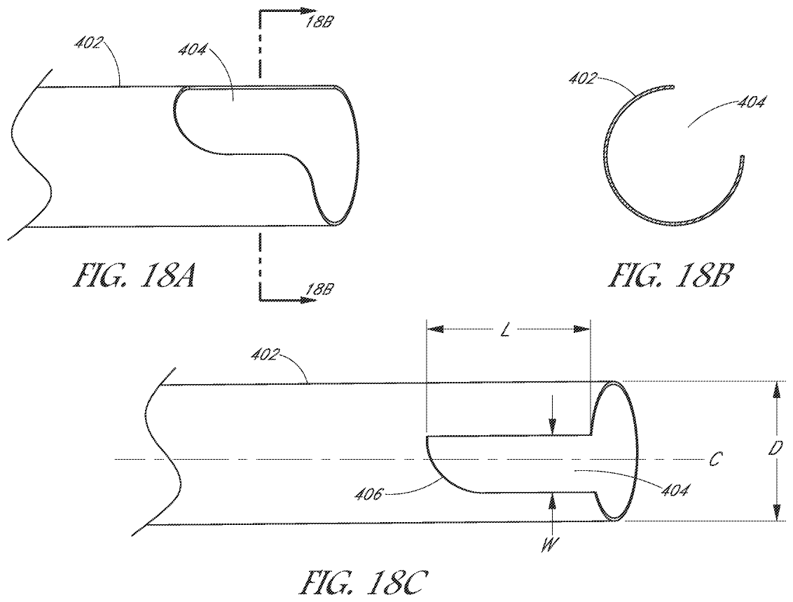

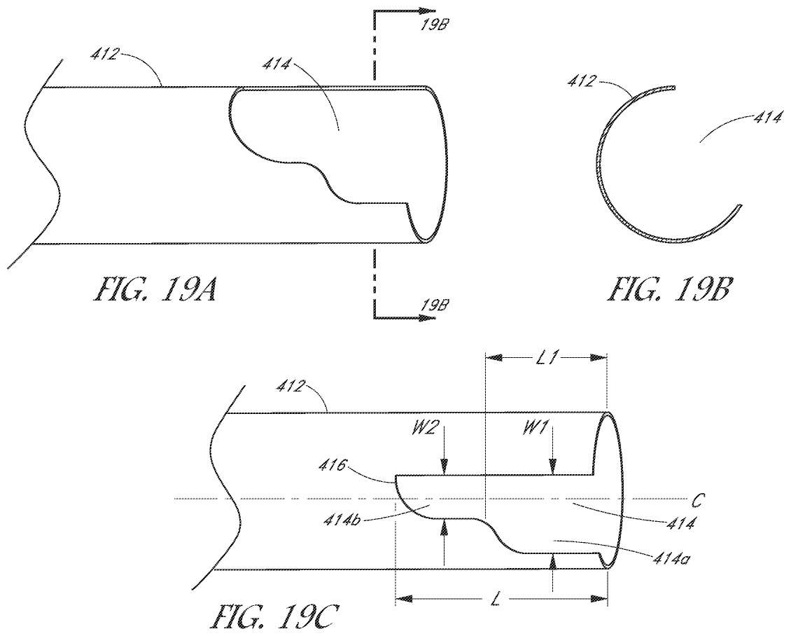

FIGS. 19A and 19C are a perspective view and a top view of an end portion of another embodiment of a cannula 412 that can be used with any embodiments of the devices disclosed herein. FIG. 19B is a section view of the embodiment of the cannula 412 shown in FIG. 19A, taken through line 19B-19B of FIG. 19A. As shown, the cannula 412 can have a notch or cutout 414 formed in an end portion of the cannula 412 which can assist in the deployment and/or retraction or collapse of any of the applicator portion embodiments disclosed herein. With reference to FIG. 18C, in some embodiments, the cutout 414 can have an asymmetric shape about a plane extending through an axial centerline C of the cannula 412—for example and without limitation, through an plane that is through the axial centerline C and is perpendicular to the plane of the figure (e.g., perpendicular to the plane of the paper).

Additionally, in any embodiments, the notch 414 can be a double notch having a first portion 414a and a second portion 414b that is narrower than the first portion 414a. For example and without limitation, the first portion 414a can have a width W1 that can be approximately double the width W2 of the second portion 414b, or can be from 50% greater (or approximately 50% greater) to 100% greater (or approximately 100% greater) than the width W2 of the second portion 414b. In any embodiments, the first portion 414a of the notch 414 can have a length L1 that is 50% (or approximately 50%) of the overall length L of the notch 414, or a length L1 that is from 30% (or approximately 30% or less than 30%) to 80% (or approximately 80% or less than 80%) of the overall length L of the notch 414, or a length L1 that is from 40% (or approximately 40%) to 60% (or approximately 60% or less than 60%) of the overall length L of the notch 414. In some embodiments, the notch 414 can terminate in a curved end portion 416.

FIG. 21 is a top view of another embodiment of a device 450 having an applicator 456 that can be used with any device embodiments disclosed herein. The device 450 and/or applicator 456 can include any of the components, features, materials, and/or other details of any of the other device or applicator embodiments disclosed herein, including without limitation device 100 and/or applicator 106 described above, in combination with or in place of any of the features described below to form new embodiments. With reference to FIG. 21, the applicator 456 can have an applicator portion 460 that is coupled with a core member or elongate body 454 that extends through at least part (or all) of the device 450.

With reference to FIG. 25, which is a section view of the embodiment of the applicator portion 460 that can be used with any device embodiments disclosed herein, taken through line 25-25 of FIG. 21 so that the section view is perpendicular to the axial centerline of the applicator 456. As shown, some embodiments of the applicator portion 460 can have a symmetrical shape about center plane C and can have a generally uniform thickness about the entire width of the applicator portion 460.

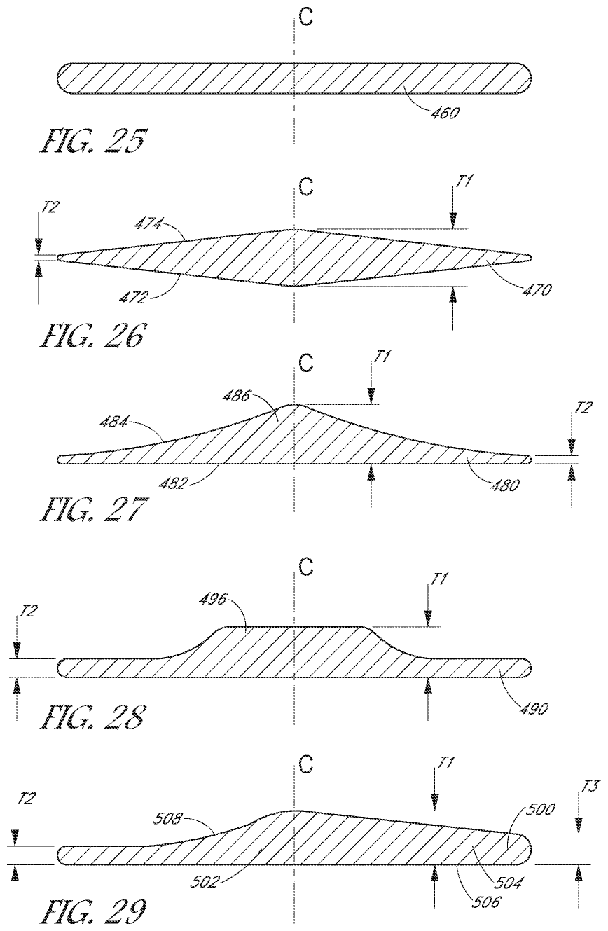

FIG. 26 is a section view of another embodiment of an applicator portion 470 that can be used with any device embodiments disclosed herein, wherein the section view is perpendicular to the axial centerline of applicator. As shown, some embodiments of the applicator portion 470 can have a symmetrical shape about center plane C and can have a tapering thickness wherein the applicator portion 470 tapers from a first thickness T1 in a middle portion of the applicator portion 470 to a second, lesser thickness T2 at one or both of the lateral sides of the applicator portion 470. In some embodiments, as shown in FIG. 26, a bottom surface 472 and/or a top surface 474 of the applicator portion 470 can be angled from the center portion of the applicator portion 470 toward the lateral edges of the applicator portion 470. In other embodiments, as shown in FIGS. 27 and 28, the bottom surface 472 and/or the top surface 474 of the applicator portion 470 can be generally planar from the center portion of the applicator portion 470 toward the lateral edges of the applicator portion 470.

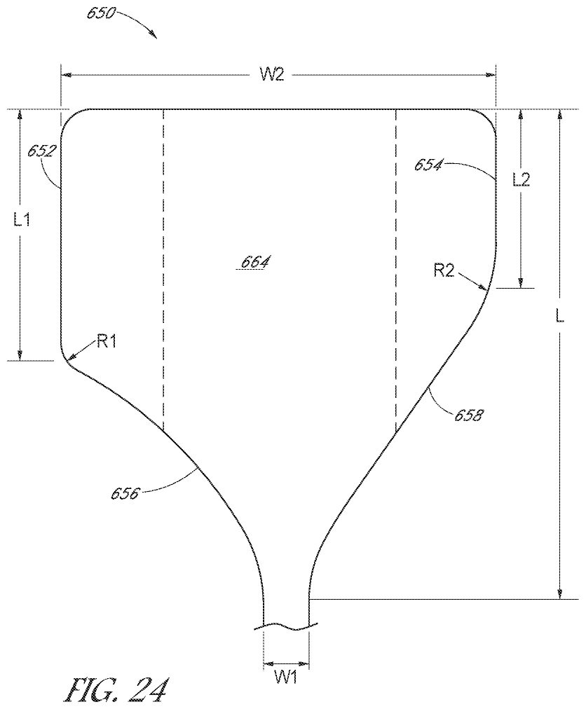

In any embodiments disclosed herein, a bottom surface and/or a top surface of the applicator portion can be concave or curved from the center portion of the applicator portion 470 toward the lateral edges of the applicator portion 470. For example and without limitation, as shown in FIG. 27, a top surface 484 of the applicator portion 480 can be curved from the center portion of the applicator portion 470 toward the lateral edges of the applicator portion. In any embodiments disclosed herein, the center portion, having an increased thickness (such as thickness T1), can be narrow, as with center portion 486 of the embodiment of the applicator portion 480 shown in FIG. 27, or can be wider, as with center portion 496 of the embodiment of the applicator portion 490 shown in FIG. 28 or the center portion 664 of the embodiment of the applicator portion 650 shown in FIG. 24. For example and without limitation, the center portion can have a width that is 30% (or approximately 30%) of the width of the entire applicator portion, or from 10% (or approximately 10% or less than 10%) to 60% (or approximately 60% or more than 60%), or from 20% (or approximately 20%) to 40% (or approximately 40%) of the width of the entire applicator portion.

In some embodiments, the first thickness T1 can be 0.2 mm (or approximately 0.2 mm), or from 0.1 mm (or approximately 0.1 mm or less than 0.1 mm) to approximately 1 mm (or approximately 1 mm or greater than 1 mm), or from 0.15 mm (or approximately 0.15 mm) to approximately 0.5 mm (or approximately 0.5 mm), or of any value or from and to any values within these ranges. In any embodiments, the second thickness T2 can be 0.1 mm (or approximately 0.1 mm), or from 0.05 mm (or approximately 0.05 mm or less than 0.05 mm) to approximately 0.5 mm (or approximately 0.5 mm or greater than 0.5 mm), or from 0.075 mm (or approximately 0.075 mm) to approximately 0.25 mm (or approximately 0.25 mm), or of any value or from and to any values within these ranges.

In some embodiments, the first thickness T1 can be 50% (or approximately 50%), or from 10% (or approximately 10% or less than 10%) to approximately 80% (or approximately 80% or greater than 80%), or from 20% (or approximately 20%) to approximately 60% (or approximately 60%), or from 30% (or approximately 30%) to approximately 40% (or approximately 40%), or of any value or from and to any values within these ranges.

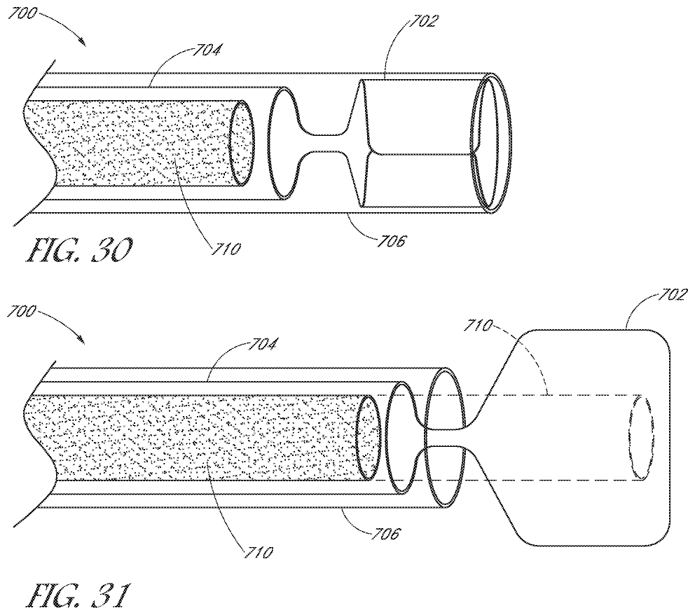

Further, in any embodiments disclosed herein, the applicator portion 470 can have an asymmetrical cross section such that a first side of the applicator portion 470 has a different shape and/or thickness as compared to a shape and/or thickness of a second side portion 470, wherein the first and second side portions can be defined as being divided by the centerline axis C. For example, as shown in FIG. 29, which is a section view of another embodiment of an applicator portion 500 that can be used with any device embodiments disclosed herein, a first side portion 502 can have a first shape and thickness profile that is different than a second shape and thickness profile of a second side portion 504 of the applicator portion 500. The thickness T1 at a center portion can be greater than a thickness T2 at a lateral edge or side portion of the first side portion 502, and a thickness T3 at a lateral edge or side portion of the second side portion 504 can be greater than the thickness T2 at the lateral edge or side portion of the first side portion 502. Additionally, in any embodiments disclosed herein, the shape of the bottom surface 506 can be different than the shape of the top surface 508, and the shape of the bottom surface 506 on the first side portion 502 can be the same or different than the shape of the bottom surface 506 on the second side portion 504, and/or the shape of the top surface 508 on the first side portion 502 can be the same or different than the shape of the top surface 508 on the second side portion 504.

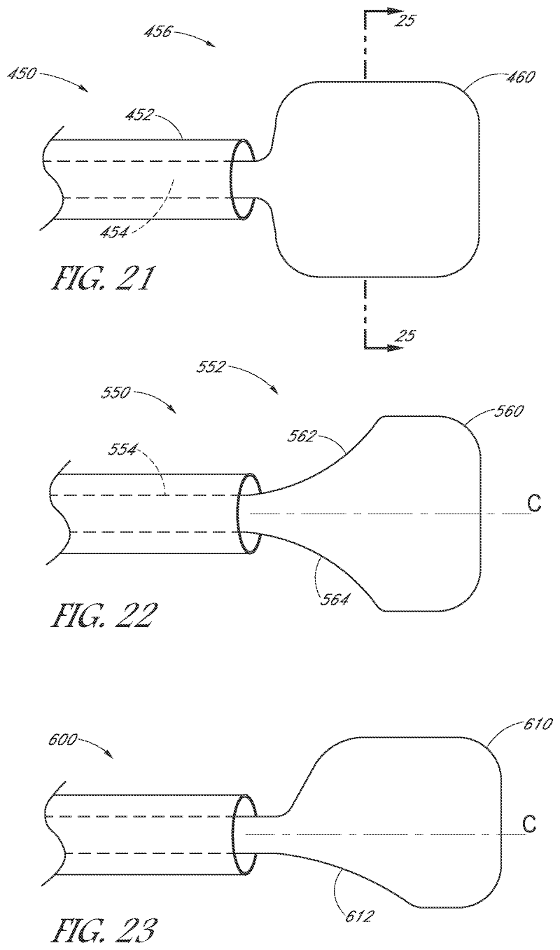

FIG. 22 is a top view of another embodiment of a device 550 having an applicator 552 that can be used with any device embodiments disclosed herein. The device 550 and/or applicator 556 can include any of the components, features, materials, and/or other details of any of the other device or applicator embodiments disclosed herein, including without limitation devices 100, 450 and/or applicators 106, 456 described above, in combination with or in place of any of the features described below to form new embodiments. With reference to FIG. 22, the applicator 552 can have an applicator portion 560 that is coupled with a core member or elongate body 554 that extends through at least part (or all) of the device 550. With reference to FIG. 22, the applicator portion 560 can have a first tapering transition portion 562 and a second tapering transition portion 564. The first and second tapering transition portions can assist in collapsing, folding, rolling, furling, or otherwise contracting the applicator portion upon retraction of the applicator portion into the cannula. In some embodiments, the first and second tapering transition portions 562, 564 can be symmetrical about the centerline C of the applicator portion 460. In other embodiments, the applicator portion can have tapering transition portions on both sides of the applicator portion, but which are sized and/or shaped differently, such as with the first and second tapering transition portions 656, 658 of the embodiment of the applicator portion 650 shown in FIG. 24. For example and without limitation, a first tapering transition portion can be longer than a second tapering transition portion. In other embodiments, the applicator portion can have just one tapering transition portion, such as with the applicator portion 610 of the embodiment of the device 600 illustrated in FIG. 23.

With reference to FIG. 24, in any embodiments, the applicator portion (such as applicator portion 650) can be configured such that a length L1 of a first side wall portion (such as side wall portion 652, which is also referred to herein as a first side portion) can be shorter than an overall length L of the applicator portion due to the presences of a first tapering transition portion (such as first tapering transition portion 656). For example, the length L1 of the first side wall portion can be approximately 40% of the overall length L of the applicator portion, or from or from approximately 30% to approximately 50% of the overall length of the applicator portion.

Further, a length L2 of the second side wall (such as side wall portion 654, which is also referred to herein as a second side portion) can be shorter than the length L1 of the first side wall. This can be due to a different length, angle, and/or other shape of the second tapering side wall portion (such as second tapering side wall portion 658) and/or a larger radius at the intersection of the second wall portion and the second tapering side wall portion. The radius at the intersection of the second wall portion and the second tapering side wall portion (such as radius R2 in FIG. 24) can be significantly greater (for example, from 2 to 20 times or more greater, or from 5 to 10 times greater, or any values or ranges of values within the foregoing ranges) than a radius at the intersection of the first wall portion and the first tapering side wall portion (such as radius R1 in FIG. 24). In any embodiments, the second side transition portion 658 can be more gradual than the first side transition portion 656, to facilitate the collapse or contraction of the applicator portion.

In some embodiments, the first side portion 652 can have a first transition portion that has a first length in the axial direction and the second side portion 654 can have a second transition portion that has a second length in the axial direction that is greater than the first length of the first transition portion. In some embodiments, the shorter length of the first transition portion can cause the first side portion 652 of the applicator tip 650 to collapse when the applicator portion 650 is withdrawn into the outer sleeve of the device before the second side portion 654 of the applicator portion 650 collapses. This can, in some embodiments, allow the applicator portion 650 to collapse to a more compact size compared with a symmetrical applicator portion.

FIG. 30 is a perspective view of an end portion of another embodiment of a delivery device 700, showing an applicator 702 that is coupled with an inner cannula 704 in a retracted and collapsed state within the outer cannula body or sleeve 706 and a substance delivery cannula 710 in a retracted state within the outer cannula body 706. FIG. 31 is a perspective view of the end portion of the embodiment of a delivery device 700 shown in FIG. 30, showing the applicator 702 in a partially advanced position and expanded state. FIG. 31 also shows that the delivery cannula 710 is, in some embodiments, axially extendable so as to extend to a distal end portion of the applicator portion 702 and retractable while the substance is being exuded from the substance delivery cannula 710. The device 700 can be configured to limit a range of extension of the substance delivery cannula 710 so that the substance delivery cannula does not extend beyond a distal end of the applicator portion 702. The device 700 can also be configured so that the substance delivery cannula 710 is in a fixed position at or near the end of the sleeve 706, allowing delivery of substance onto applicator 702 surface as applicator 702 is advanced beyond an end of outer cannula or sleeve 706. In some embodiments, the only movable or extendable component of the device or a distal end portion of the device can be the applicator and applicator portion, which can be integrally connected.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A device for applying a bioadhesive substance to a retina, the device comprising:
an outer sleeve comprising a proximal end, a distal end, a passageway extending along a length of the outer sleeve, and an opening at the distal end of the outer sleeve;
an applicator tip comprising:
an elongate body having a proximal end and a distal end;
an applicator portion coupled with the distal end of the elongate body;
a substance supply lumen extending along a length of the device through which a bioadhesive substance can pass so that the bioadhesive substance can be applied to at least the applicator portion of the applicator tip; and
a source of the bioadhesive substance in fluid communication with at least the substance supply lumen;
wherein:
the applicator tip is configured to move between a first position wherein the applicator portion is contained within the outer sleeve and a second position wherein the applicator tip is positioned past the opening at the distal end of the outer sleeve; and
the applicator portion has a first width when the applicator portion is in the first position and a second width when the applicator portion is the second position, the second width being substantially greater than the first width.

2. The device of claim 1, wherein the substance supply lumen is a passageway extending through at least the elongate body of the applicator tip.

3. The device of claim 1, wherein the substance supply lumen comprises a separate cannula positioned within the outer sleeve and extendable and retractable relative to the outer sleeve.

4. The device of claim 1, wherein the substance supply lumen is axially fixed relative to the outer sleeve and has an opening that is adjacent to an end portion of the outer sleeve such that the bioadhesive substance can be exuded onto the applicator portion along at least a portion of a length of the applicator portion as the applicator portion is advanced distally.

5. The device of claim 1, wherein the applicator portion is biased such that a first side portion rolls or folds toward a second side portion of the applicator portion when the applicator tip is withdrawn in the proximal direction into the outer sleeve.

6. The device of claim 1, wherein the passageway through at least the elongate body of the applicator tip is in communication with a plurality of openings passing through a first surface of the applicator portion.

7. The device of claim 1, wherein the applicator portion has an asymmetric shape such that a first side portion on one side of an axial centerline of the applicator portion has a different shape than a second side portion on an opposite side portion of the applicator portion, the first and second sides being divided an axial centerline of the applicator portion.

8. The device of claim 1, wherein the applicator portion has an asymmetric shape having a first side portion on one side of an axial centerline of the applicator portion that has a first length that is longer than a second length of a second side portion on a second side of the axial centerline of the applicator portion.

9. The device of claim 8, wherein the first side portion has a first transition portion that has a first length in the axial direction and the second side portion has a second transition portion that has a second length in the axial direction, and wherein the first length of the first transition portion is less than the second length of the second transition portion.

10. The device of claim 1, wherein the applicator portion has an asymmetric width.

11. The device of claim 1, wherein the applicator portion has an asymmetric thickness in a widthwise direction, such that a thickness profile of the applicator portion on a first side of the applicator portion is different than a thickness profile of the application portion on a second side of the applicator portion.

12. The device of claim 11, wherein the first side portion is more flexible than the second side portion of the applicator portion.

13. The device of claim 1, wherein the applicator portion is configured such that a first side portion contracts before a second side portion contracts when the applicator portion is withdrawn into the outer sleeve.

14. The device of claim 1, wherein the applicator portion has an asymmetric width and an asymmetric thickness.

15. The device of claim 1, wherein the outer sleeve has a notch in the distal end thereof.

16. A device for applying a substance to a tissue surface, the device comprising:
a cannula comprising a proximal end, a distal end, and a passageway extending along a length of the cannula; and
an applicator tip comprising:
an elongate body having a proximal end and a distal end;
an applicator portion coupled with the distal end of the elongate body;
a passageway through at least the elongate body of the applicator tip;
a handle coupled with a proximal end of the cannula; and
a source of the substance in fluid communication with at least the passageway extending along a length of the elongate body of the applicator tip;
wherein:
the applicator tip is configured to move along a length of the passageway of the cannula between a first position wherein the applicator portion is contained within the cannula and a second position wherein the applicator tip extends past the distal end of the cannula;
the applicator portion has a first width when the applicator portion is in the first position and a second width when the applicator portion is the second position, the second width being substantially greater than the first width; and
the applicator portion is biased to collapse within the passageway of the cannula when the applicator tip is withdrawn in the proximal direction into the cannula.

17. The device of claim 16, wherein the substance is a bioadhesive.

18. The device of claim 16, wherein the applicator portion is biased such that a first side portion rolls or folds toward a second side portion of the applicator portion when the applicator tip is withdrawn in the proximal direction into the cannula.

19. The device of claim 16, wherein the passageway through at least the elongate body of the applicator tip is in communication with an opening at a proximal end of the applicator portion.

20. A method of repairing a defect in a retinal tissue, comprising:
advancing a device for applying a bioadhesive substance toward the defect;
covering at least a portion of an applicator tip of the device with the bioadhesive substance;
advancing the applicator tip past a distal end of an outer sleeve of the device; and
transferring the bioadhesive substance from the applicator tip of the device to the defect.
